(19) United States
(12) Patent Application Publication
(10) Pub. No.: US 2022/0339236 A1
(43) Pub. Date: Oct. 27, 2022

(54) TREATMENT OF GENETIC DISEASES CHARACTERIZED BY UNSTABLE mRNAs

(71) Applicant: Yissum Research Development Company of the Hebrew University of Jerusalem Ltd., Jerusalem (IL)

(72) Inventors: Rotem KARNI, Jerusalem (IL); Adi AMAR-SCHWARTZ, Jerusalem (IL)

(73) Assignee: Yissum Research Development Company of the Hebrew University of Jerusalem Ltd., Jerusalem (IL)

(21) Appl. No.: 17/762,504

(22) PCT Filed: Sep. 23, 2020
(86) PCT No.: PCT/IL2020/051038

§ 371 Date: Mar. 22, 2022

Related U.S. Application Data
(60) Provisional application No. 62/904,242, filed on Sep. 23, 2019.

(51) Int. Cl. A61K 38/00; A61P 35/00; A61K 47/00

(52) U.S. Cl. CPC A61K 38/005; A61P 35/00; A61K 47/00

(57) ABSTRACT

Methods of treating a disease characterized by mRNA instability or nonsense-mediated decay of an mRNA of a disease-associated gene in a subject by administering a pharmaceutical composition comprising at least one agent that decreases FTO expression, function or both are provided. Kits and pharmaceutical compositions comprising an agent that decreases FTO expression, function or both and a read-through promoting agent are also provided, as are methods of determining suitability of a subject to be treated with an agent that decreases FTO expression, function or both.

### CROSS REFERENCE TO RELATED APPLICATIONS

This application claims the benefit of priority of U.S. Provisional Patent Application No. 62/904,242, filed Sep. 23, 2019, the contents of which are all incorporated herein by reference in their entirety.

### FIELD OF INVENTION

The present invention is in the field of mRNA instability therapy.

### BACKGROUND OF THE INVENTION

The nonsense-mediated mRNA decay mechanism (NMD) is an evolutionarily conserved translation-dependent mechanism, in all eukaryotes, that is responsible for recognizing and eliminating aberrant messenger RNA (mRNA) transcripts to prevent the production of truncated peptides that could have toxic and detrimental effects on the cell. NMD plays a critical role in preventing the potential dominant-negative effect of non-functional proteins within the cell, as well as the prevention of misfolded protein accumulation and subsequent initiation of the ER stress response.

NMD primarily protects the cell against the deleterious effects of premature termination codons (PTCs), but there is a growing body of evidence that mutation-, codon-, gene-, cell-, and tissue-specific differences in NMD efficiency can alter underlying disease pathology. In fact, there is evidence that in certain genetic disorders, NMD can act to aggravate disease pathology and worsen the clinical phenotype, because degradation of the mutated mRNA prevents translation and accumulation of truncated peptides that retain residual activity.

Additional mechanisms of mRNA degradation were discovered recently in which RNA methylation of adenine on position 6 (m6A) modulates mRNA stability. In specific the presence of the methylation mark enhanced RNA degradation. The methylated mRNA is identified by a “reader” protein that recognizes the m6A and recruits RNA nucleases to degrade the mRNA or affect its stability indirectly. Thus, increased m6A is known to be associated with mRNA instability.

There are genetic disorders in which truncated peptides retain function, such as Becker muscular dystrophy (BMD), in which mRNA degradation acts to worsen the disease phenotype by removing even this limited functional capability. Duchenne muscular dystrophy (DMD) is a genetic disorder caused by mutations in the dystrophin gene. Dystrophin gene transcripts carrying mutations that are NMD-insensitive produce truncated peptides with residual activity that can yield Becker muscular dystrophy (BMD), a milder form of DMD.

Aberrant RNA splicing is a hallmark of cancer and is specifically pronounced in cancers harboring mutations in splicing factors. Tumors harboring splicing factor mutations (e.g. SF3B1, U2AF1, U1, SRSF2 and others) or tumors with mutations in their DNA repair machinery (e.g. MSH2, MSH6, MLH1, ERCC1, ERCC4, MBD4, BRCA1, BRCA2, Rad51 and others) produce many aberrant transcripts containing premature termination codons (PTCs). These PTC containing transcripts if expressed would produce truncated proteins which will be toxic to the cancer cells, however, the PTC containing transcripts are degraded by NMD. Thus, tumors harboring splicing factor mutations or mutations in DNA repair factors are dependent on efficient NMD to get rid of these harmful transcripts and are hyper-sensitive to inhibition of NMD.

In many genetic diseases, nonsense mutations, generating PTC, cause degradation of the mRNA and the affected gene does not produce protein. In such cases, if inhibition of NMD stabilizes the mRNA, another drug that suppresses the PTC and enables translation by the ribosome through the PTC is required to alleviate the disease.

Diseases in which a mutation creates a PTC in the affected gene will benefit from a combination of mRNA stabilizing compounds and nonsense suppression therapy. The goal of nonsense suppression therapy is to exploit a natural process and enhance read-through by allowing near-cognate aminoacyl-tRNAs to out-compete the release factor complex and enter the ribosomal A site. By recoding the PTC into a sense codon, sufficient full-length, and possibly functional, protein may be produced to provide a therapeutic benefit to patients with the genetic disease. Read-through compounds will bind to either the 40S or 60S subunit of the ribosome and decrease the fidelity of ribosome pausing at the PTC. The purpose of nonsense suppression therapy is to trick the ribosome into accepting near-cognate aminoacyl-tRNAs into the A-site, therefore enhancing natural PTC read-through and increasing the abundance of full-length protein.

There are several compounds that can induce read-through of PTCs, such as aminoglycosides, modified aminoglycosides (NB30, NB54, NB84), ataluren (PTC124), Amlexanox, erythromycin, azithromycin and RTC13. These compounds have been shown in both in vitro and in vivo models to alleviate disease pathogenesis by enhancing PTC read-through. However, the efficacy of these compounds is hampered by mRNA degradation caused by NMD.

A few disorders have been heavily studied using nonsense suppression therapy and these studies have proceeded into clinical trials. These diseases include CF, BMD/DMD, factor VII deficiency, Hailey-Hailey disease, hemophilia A and hemophilia B, leucocyte adhesion deficiency 1 (LAD1) and McArdle disease.

Aberrant mRNA degradation or decay may be the result of aberrations other than nonsense mutations, such as in-frame mutations, deletions or insertions, causing a non-functional/partially functional protein. BMD is an example of such a disease. Even though some BMD patients show severe symptoms, similar to DMD, no targeted therapy is aimed to specifically treat BMD.

FTO was identified as the first RNA demethylase that catalyzes oxidative demethylation of N6-methyladenosine (m6A) on mRNA. FTO-mediated m6A demethylation has been found to regulate many biological processes including preadipocyte differentiation, heat shock stress induced cap-independent translation, UV-induced DNA damage and acute myeloid leukemia (oncogenic FTO). Inhibitors of FTO were suggested as a strategy to oncogenic FTO cancers, as well as obesity and pathological conditions related to bone-mineral density disorders.

The use of non-steroidal anti-inflammatory drugs (NSAIDs) has been proposed for the treatment of muscular dystrophies (MD). One FTO inhibitor, Meclofenamic acid, is also a known NSAID. However, beyond its anti-inflammatory effect no link has been made between its FTO inhibitory function and MD treatment. New methods and compositions for the treatment of diseases characterized by aberrant RNA degradation and thus greatly needed.

### SUMMARY OF THE INVENTION

The present invention provides methods of treating a disease characterized by mRNA instability or nonsense-mediated decay of an mRNA of a disease-associated gene in a subject by administering a pharmaceutical composition comprising at least one agent that decreases FTO expression or function. Kits and pharmaceutical compositions comprising an agent that decreases FTO expression or function and a read-through promoting agent are also provided, as are methods of determining suitability of a subject to be treated with an agent that decreases FTO expression or function.

According to a first aspect, there is provided a method of treating a disease characterized by mRNA instability of an mRNA of a disease-associated gene in a subject in need thereof, the method comprising administering the subject a pharmaceutical composition comprising at least one agent that inhibits fat mass and obesity associated protein (FTO) expression or function, wherein the agent is not a non-steroidal anti-inflammatory drug (NSAID), thereby treating the disease.

According to another aspect, there is provided a method of treating a disease characterized by nonsense mediated decay (NMD) of an mRNA of a disease-associated gene in a subject in need thereof, the method comprising administering to the subject a pharmaceutical composition comprising at least one agent that inhibits FTO expression or function, thereby treating the disease.

According to another aspect, there is provided a pharmaceutical composition comprising at least one agent that decrease FTO expression, function or both, at least one read-through promoting agent and a pharmaceutically acceptable carrier.

According to another aspect, there is provided a kit comprising at least one agent that decreases FTO expression, function or both and at least one read-through promoting agent.

According to another aspect, there is provided a method of determining suitability of a subject suffering from a disease to be treated with an agent that decreases FTO expression, function or both, the method comprising measuring mRNA stability of an mRNA of a gene associated with the disease in the subject, wherein determining instability of the mRNA indicates the subject is suitable for treatment with the agent.

According to some embodiments, the agent is not an NSAID.

According to some embodiments, the agent is not meclofenamic acid.

According to some embodiments, the agent is not a derivative of meclofenamic acid.

According to some embodiments, the derivative of meclofenamic acid is selected from Mefenamic acid, Niflumic acid, and Flufenamic acid.

According to some embodiments, the agent is not an isooxazoline derivative.

According to some embodiments, the agent is a small molecule FTO inhibitor.

According to some embodiments, the agent is a nucleic acid molecule that inhibits FTO transcription, inhibits FTO translation, induces FTO mRNA degradation or alters the FTO genetic locus.

According to some embodiments, the agent is an FTO inhibitor selected from the group consisting of: Meclofenamic acid, Mefenamic acid, Niflumic acid, Flufenamic acid, 2-(2-toluidino)benzoic acid (2TBA); 2-(3-toluidino)benzoic acid (3TBA); 4-chloro-2-[3-(trifluoromethyl)anilino]benzoic acid (CTB); 5H-Dibenz[b,f]azepine (5HD), Clonixin, 10H-Dibenz[b,f]azepine (10HD), and methyl 10,11-dihydro-5H-dibenzo[b,f]azepine-4-carboxylate (MDB).

According to some embodiments, the non-NTHE agent is an FTO inhibitor selected from the group consisting of: 2-(2-toluidino)benzoic acid (2TBA); 2-(3-toluidino)benzoic acid (3TBA); 4-chloro-2-[3-(trifluoromethyl)anilino]benzoic acid (CTB); 5H-Dibenz[b,f]azepine (5HD), Clonixin, 10H-Dibenz[b,f]azepine (10HD), methyl 10,11-dihydro-5H-dibenzo[b,f]azepine-4-carboxylate (MDB).

According to some embodiments, the FTO inhibitor is selected from MDB, 2TBA, 3TBA and 5HD.

According to some embodiments, a disease-associated gene is a disease-causing gene.

According to some embodiments, mRNA instability comprises aberrant mRNA degradation.

According to some embodiments, the disease is further characterized by the presence of a premature termination codon.

According to some embodiments, the disease is selected from a muscular dystrophy characterized by mRNA instability or NMD of a disease-associated gene and cancer characterized by mRNA instability or NMD of a disease-associated gene.

According to some embodiments, the disease is selected from the group consisting of: muscular dystrophy, cystic fibrosis, Ullrich disease, factor VII deficiency, Hailey-Hailey disease, hemophilia A, hemophilia B, leucocyte adhesion deficiency 1 (LAD1), cancer, McArdle disease, obesity and pathological conditions related to bone-mineral density disorders.

According to some embodiments, the muscular dystrophy is selected from Bechet's muscular dystrophy, and Duchenne muscular dystrophy, and the cancer is selected from lung cancer and acute myeloid leukemia (AML).

According to some embodiments, the cancer does not comprise oncogenic FTO expression.

According to some embodiments, the cancer comprises a mutation of a splicing factor gene or a DNA repair gene, optionally wherein the DNA repair gene is a mismatch repair (MMR) gene.

According to some embodiments, the method further comprises confirming mRNA instability or NMD of the mRNA of the disease-associated gene before the administering.

According to some embodiments, the method further comprises administering at least one read-through promoting agent.

According to some embodiments, the read-through promoting agent is selected from the group consisting of: aminoglycosides, modified aminoglycosides, erythromycin, azithromycin, (5Z)-2-Amino-5-[[5-(2-nitrophenyl)-2-furanyl]methylene]-4(5H)-thiazolone (RTC13), 3-[5-(2-Fluorophenyl)-1,2,4-oxadiazol-3-yl]benzoic acid (Ataluren), and 2-amino-7-isopropyl-5-oxo-5H-chromeno[2,3-b]pyridine-3-carboxylic acid (Amlexanox).

According to some embodiments, the measuring mRNA stability comprises measuring NMD in the subject and wherein detecting NMD indicates the subject is suitable for treatment.

According to some embodiments, the method comprises receiving a sample from the subject and measuring mRNA stability in the sample.

According to some embodiments, the disease is cancer, and the gene is an anti-cancer gene.

According to some embodiments, the disease is a muscular dystrophy, and the gene is a muscle promoting gene.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### DESCRIPTION OF FIGURES

FIG. 1. Scheme of the dystrophin gene. Exons whose nucleotide length is divisible by three are depicted as rectangles. Exons whose length is not divisible by three, causing a shift in the reading frame of the dystrophin protein when deleted, are depicted by a different shape. Location of mutations found in patient samples collected as skin biopsies are marked by asterisks/hash and different colors; black, green, orange for nonsense mutations (asterisks); red for deleted exons (hash). Summary of the number of each type of sample collected is listed on the right side.

FIGS. 2A-B. Dystrophin mRNA is degraded by NMD in DMD patient-derived skin fibroblasts. 2A. Q-RT-PCR of dystrophin mRNA levels from DMD patient-derived skin fibroblasts using primers for exons 65-67 of the dystrophin gene. 2B. Q-RT-PCR of dystrophin mRNA levels in DMD patient-derived skin fibroblasts, before and after exposure to cycloheximide (CHX) for 24 hours, using primers for exons 65-67 of the dystrophin gene.

FIG. 3. Drugs that inhibit RNA degradation stabilize NMD-prone mRNAs. HeLa cells were exposed to NMD inhibitors (amlexanox, 5-azacytidine) and FTO inhibitor (Meclofenamic acid) and its analogs (Mefenamic acid, Flufenamic acid or Niflumic acid) for 72 hours. The mRNA levels of known NMD targets (ATF3 and RPL3) were measured using Q-RT-PCR.

FIGS. 4A-B. Inhibition of NMD elevates NMD-prone transcripts of SR proteins. Primary skin fibroblasts from four patients with nonsense mutations in the dystrophin gene were exposed to either 5-AzaC alone or 5-AzaC in combination with Ataluren (PTC124) for 72 hours. (4A) mRNA levels of SR proteins, known NMD targets, were measured using RT-PCR. (4B) Bar charts quantifying the results provided in 4A.

FIGS. 5A-B. Combination of NMD inhibitor+Ataluren (PTC124) elevates the level of SR proteins in patient fibroblasts. Primary skin fibroblasts from four patients with nonsense mutations in the dystrophin gene were exposed to either 5-AzaC alone or 5-AzaC in combination with Ataluren (PTC) for 72 hours. (5A) Western blots of protein levels of SR proteins, and known NMD targets. (5B) Bar charts quantifying the results provided in 5A.

FIGS. 6A-G. Compounds used in the study (I). 6A. Meclofenamic acid was shown to act as a FTO inhibitor (PMID: 25452335). Compounds shown in 6A, 6B, 6D, 6E were tested as Meclofenamic acid analogs and potential FTO inhibitors. 6C. Ataluren is a read-through drug that enables translation through nonsense mutations. 6F. 5′-Azacytidine is a drug for the treatment of MDS/AML and was shown to inhibit NMD. 6G. Amlexanox is an approved drug (in Japan) for inflammation and was shown to both inhibit NMD and enable read-through of nonsense mutations.

FIGS. 7A-G. Compounds used in the study (II). 7A. 2-(2-toluidino)benzoic acid (2TBA), 7B. 2-(3-toluidino)benzoic acid (3TBA). 7C. 4-chloro-2-[3-(trifluoromethyl)anilino]benzoic acid (CTB). 7D. methyl 10,11-dihydro-5H-dibenzo[b,f]azepine-4-carboxylate (MDB). 7E. Clonixin (Clo). 7F. Flunixin Meglumine (Flun). 7G. 5H-Dibenz[b,f]azepine (5HD).

FIGS. 8A-D. The effects of drugs, which inhibit NMD or FTO, on the stability of NMD-prone transcripts and DMD mRNA in HeLa, HEK293 and DMD patient-derived cells. (8A) RT-PCR of NMD-prone transcripts in HeLa or HEK293 cells treated with the indicated compounds for 48 hours. (8B) Bar charts quantifying the results provided in 8A. (8C) RT-PCR of NMD-prone transcripts and dystrophin mRNA (dp71) in DMD patient-derived fibroblasts, with the indicated nonsense mutations in the dystrophin gene, treated with the indicated compounds for 48 hours. (8D) Bar charts quantifying the results provided in 8C.

FIGS. 9A-B. The effect of drugs, which inhibit NMD or FTO, on the stability of dystrophin mRNA in DMD patient-derived fibroblasts. 9A-B. Primary skin fibroblasts of a DMD patient with a nonsense mutation in exon 53 of the dystrophin gene were exposed to the indicated compounds for 48 or 72 hours. Dystrophin mRNA levels (9A-B) were measured using Q-RT-PCR.

FIGS. 10A-B. Drugs that inhibit RNA degradation stabilize SRSF6 and ATF3 mRNAs in DMD patient-derived fibroblasts. 10A-B. Primary skin fibroblasts of a DMD patient with a nonsense mutation in exon 53 of the dystrophin gene were exposed to the indicated compounds for 48 or 72 hours. mRNA of known NMD targets, SRSF6 (10A) and ATF3 (10B), were measured using Q-RT-PCR.

FIGS. 11A-D. Trans-differentiation of skin fibroblasts from a healthy male into myocytes by MyoD induction. 11A. Primary skin fibroblasts from a healthy male were transduced with Tet-on inducible MyoD lentiviruses for 24 hours. 48 hours after infection cells were seeded on matrigel coated dishes (2.5×10⁵ cells/well) and exposed to doxycycline (3 ug/ul) for 11 days. Doxycycline was changed every 2 days. Dystrophin protein levels were measured using western blot analysis. Lysates from mouse tibia anterior (K-4-TA) muscle is shown as a positive control. 11B-C. Muscle differentiation markers were measured using RT-PCR (11B) and Q-RT-PCR (11C). 11D. Microscopy images were taken from cells expose to doxycycline for 11 days. DAPI straining (blue), mCherry staining indicates expression of MyoD (red).

FIGS. 12A-B. MDB and Amlexanox (Amx) stabilized dystrophin mRNA and protein in differentiated BMD patient-derived cells. 12A-B. BMD patient-derived skin fibroblasts (del 45-49) were infected with Tet-on inducible MyoD viruses for 24 hours. 48 hours after transduction cells were seeded on matrigel coated dishes (2.5×10⁵ cells/well) and exposed to doxycycline (3 ug/ul) for 11 days. On day 6 of differentiation cells were exposed to various compounds (mec 10 μm, 5-AzaC 4 μm, amx 5 μm, 2TBA 25 μm, 3TBA 25 μm or MDB 10 μm). Muscle differentiation markers and dystrophin mRNA were measured using RT-PCR (12A). Dystrophin protein levels were measured by western blot analysis (12B). Asterisk marks a non-specific band. Arrow marks dystrophin protein.

FIGS. 13A-C. Combination of FTO inhibitors with a read-through drug (Amlexanox), elevates the protein levels of SR proteins. Skin fibroblasts from a BMD patient were treated for 72 hours with the indicated concentrations of the FTO inhibitors; Meclofenamic acid (Mec), Mefenamic acid (Mef), Niflumic acid (Nif), Flufenamic acid (Flu) together with the read-through drug Amlexanox (Amx). 13A-B. The levels of SRSF1, SRSF3, SRSF6 and β-catenin (as loading control) were detected by western blot analysis. 13C. Normalized levels (to β-catenin) of each protein are shown.

FIGS. 14A-D. Knockout of FTO increases mRNA levels of NMD-prone targets. 14A. Western blot showing the expression levels of FTO in HeLa cells after transduction with CRISPR-V2 lentivirus containing FTO specific guides (KO FTO g1 and g2). 14B-D. Q-RT-PCR of known NMD prone targets (14B), transcripts containing nonsense mutations (14C) or NMD core proteins (14D) in cells described in 14A.

FIGS. 15A-C. Knockdown of FTO and UPF1 in DMD patient-derived fibroblasts containing a nonsense mutation in exon 53. 15A. Q-RT-PCR of FTO and UPF1 mRNA levels in DMD patient-derived fibroblasts containing a nonsense mutation in exon 53 following knockdown by the indicated siRNAs. 15B. Q-RT-PCR of expression levels of PTC containing mRNAs dystrophin (DP71, exons 65-66) and ATF3 normalized to GAPDH in the same cells as 15A. 15C. Western blot (left panel) of FTO protein levels and Q-RT-PCR (right panel) of several regions of dystrophin mRNA in DMD patient-derived fibroblasts containing a nonsense mutation in exon 53 transduced with CRISPR-V2 lentivirus encoding Cas9 and an FTO specific guide RNA (FTO KO) or control CRISPR-V2 lentivirus.

FIGS. 16A-C. Knockout of FTO in DMD patient-derived fibroblasts containing a nonsense mutation in exon 11. 16A-C. Western blot (16A), RT-PCR (16B) and Q-RT-PCR (16C) of DMD patient-derived fibroblasts containing a nonsense mutation in exon 11 transduced with a CRISPR-V2 lentivirus containing a FTO specific guide (FTO KO). CRISPR-V2 lentivirus containing a ALKBH5 specific guide (KO ALKBH5) was used as a control.

FIG. 17. Drug screen for FTO inhibitors that stabilize NMD-prone mRNAs. HeLa cells were treated with either DMSO or 504 or 10 μM of the noted compounds for 72 hours. After 72 hours, cells were harvested, and RNA extracted. Expression was measured by Q-RT-PCR and normalized to actin transcripts levels.

FIGS. 18A-D. MDB stabilizes dystrophin protein levels in BMD patient-derived differentiated muscle cells. 18A-B. BMD patient-derived fibroblasts (duplication exon 2-7) and healthy skin fibroblasts (1092sk) were infected with Tet-on inducible myoD viruses for 24 hours. After 48 hours infected cells were seeded on matrigel coated dishes (2.5×10⁵ cells/well) and were exposed to doxycycline (3 μg/ul) for 11 days. Doxycycline was changed every 2 days. After 5 days of differentiation cells were exposed to either MDB or 5HD alone or in combination with azithromycin (Azi) for another 6 days. Dystrophin protein levels were measured using western blot (18A) and relative quantification (18B). Arrows show dystrophin protein. 18C-D. Q-RT-PCR of muscle differentiation genes (CK2, myogenin, desmin) (18C) or DMD (18D) in cells described above.

FIGS. 19A-B. MDB and 5HD stabilize MSH6 protein levels in Ovca 433 cells. 19A-B. Western blot (top panels) and quantitation (bottom panels) of Ovca 433 cells treated with 5HD (19A) or MDB (19B) alone or in combination with Amlexanox (Amx) or Erythromycin (Ery) for 72 hours.

FIG. 20. Sensitivity of lung cancer cells (NCI-I1727) with either wild-type (NCI-GFP, NCI-U2AF1) or mutant U2AF1 (NCI-S34F, NCI-Q157R) to FTO inhibitors. Cells (2×10⁶) were seeded in 6 well plates and treated with inhibitors (5-azacytidine, MDB, 5-HD) at the indicated concentrations. Cell viability was measured after 48 hours using trypan blue viability assay.

FIG. 21. Sensitivity of leukemia cells to FTO inhibitors. Leukemia cells Kasumi 1 (AML), NKM-34F (AML harboring U2AF1 S34F mutation) and K562 (CIVIL wild-type U2AF1) were seeded in 6 well plates and treated with inhibitors (5-azacytidine, MDB, 5-HD) at the indicated concentrations. Cell viability was measured after 48 hours using trypan blue viability assay.

FIG. 22. Sensitivity of leukemia cells to FTO inhibitors. AML cell lines (Kasumi 1 and Kasumi 3) and AML cell line harboring U2AF1 S34F mutation (NKM-S34F) were treated with inhibitors (5-azacytidine, MDB, 5-HD) at the indicated concentrations. Cell viability was measured after 48 hours using trypan blue viability assay.

FIGS. 23A-G. FTO knockdown inhibits the oncogenic properties of lung cancer cells (NCI-I1727) with either wild-type (NCI-GFP, NCI-U2AF1) or mutant U2AF1 (NCI-534F, NCI-Q157R). Lung cancer cells (NCI-H727) were transduced with either wild-type (NCI-GFP, NCI-U2AF1) or mutant U2AF1 (NCI-S34F, NCI-Q157R), with or without FTO knockout. (23A) Western blot to detect FTO levels. (23B-C) Cells (1.5×10⁴) were seeded into soft agar in 6 well plates. 14 days after seeding colonies of over 100 cells were (23B) photographed and (24C) counted. (23D) Cells (2,500 cells/well) were seeded into 96 well plates. Every 24 h one plate was fixed. Cells were stained with methylene blue, washed and color was extracted and absorbance was measured by a plate reader at 650 nm. (23E-G) Cells were seeded on 6-well plates at 1000, 500 or 250 cells/well and grown for 14 days. Colonies were then fixed, stained with methylene blue and (23E) photographed, (23F) counted and (23G) color was extracted and absorbance at 650 nm was measured by a plate reader.

### DETAILED DESCRIPTION OF THE INVENTION

The present invention, in some embodiments, provides methods of treating a disease characterized by mRNA instability or nonsense-mediated decay of an mRNA of a disease-associated gene in a subject by decreasing FTO expression, function or both. Kits and pharmaceutical compositions comprising an agent that decreases FTO expression, function or both and a read-through promoting agent are also provided, as are methods of determining suitability of a subject to be treated with an agent that decreases FTO expression, function or both.

The present invention is based on the surprising finding that while the dystrophin mRNA is unstable and undergoes degradation in both DMD and BMD patients, the inhibition of the m6A de-methylation enzyme FTO, genetically or pharmacologically, stabilized dystrophin mRNA as well as other nonsense-mediated decay (NMD)-prone transcripts. It had been previously known that m6A methylation was, in some cases, a cause of mRNA instability, thus it is wholly unexpected that inhibition of an enzyme that removes the m6A mark (thereby increasing the amount of m6A present) would be able to improve mRNA stability and specifically that this inhibition would be effective in cases of NMD.

Several FTO inhibitors have been identified which stabilized dystrophin mRNA and other PTC containing transcripts. The FTO inhibitors can elevate dystrophin mRNA and protein levels in patients. Interestingly, several FTO inhibitors were found to be surprisingly effective, and indeed more effective than NSAID FTO inhibitors such as mechlofenamic acid and its derivatives.

By a first aspect, there is provided a method of treating a disease in a subject in need thereof, the method comprising decreasing fat mass and obesity associated protein (FTO) expression, function or both, thereby treating the disease.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition or method herein needs only to reduce the severity of a disease, disorder, or condition, reduce the severity of symptoms associated therewith, or provide improvement to a patient or subject's quality of life.

In some embodiments, decreasing comprises administering an agent that decreases FTO expression, function or both. In some embodiments, the administering is to a patient in need thereof. In some embodiments, the method comprises administering to a subject in need thereof an agent that that inhibits FTO expression. In some embodiments, the method comprises administering to a subject in need thereof an agent that that inhibits FTO function. In some embodiments, the method comprises administering to a subject in need thereof an agent that that inhibits FTO expression and function. In some embodiments, treating comprises administering to a subject in need thereof an agent that inhibits FTO expression or function. In some embodiments, the agent inhibits FTO expression or function. In some embodiments, the agent is a small molecule. In some embodiments, the agent is an FTO inhibitor. In some embodiments, the agent is a nucleic acid molecule. In some embodiments, the agent is specific to FTO. In some embodiments, the nucleic acid molecule is specific to FTO. As used herein, the term “specific” refers to binding to or directly modulating only FTO. A specific nucleic acid molecule will bind only to the FTO locus or mRNA and not significantly bind to another target. In some embodiments, specific is binding with at least 100% homology. In some embodiments, specific is binding with at least 95% homology. In some embodiments, specific is binding with at least 90% homology. In some embodiments, specific is binding without a mismatch. In some embodiments, specific is not decreasing expression or function of protein other than FTO.

In some embodiments, the treating comprises administering the agent. In some embodiments, the agent is a nucleic acid molecule that inhibits FTO translation. In some embodiments, the agent is a nucleic acid molecule that inhibits FTO transcription. In some embodiments, the agent is a nucleic acid molecule that induces FTO mRNA degradation. In some embodiments, the agent is a nucleic acid molecule that alters the FTO genetic locus. In some embodiments, the agent is a nucleic acid molecule that modifies the FTO genetic locus. In some embodiments, altering is deleting a portion of the locus. In some embodiments, the altering is knocking out the FTO locus. In some embodiments, altering is removing a functional FTO locus.

In some embodiments, the nucleic acid molecule is an siRNA. In some embodiments, the nucleic acid molecule is an anti-sense oligonucleotide (ASO). In some embodiments, the nucleic acid molecule is a GAPmer. In some embodiments, the nucleic acid molecule is peptide nucleic acid (PNA). In some embodiments, the nucleic acid molecule is PMO. In some embodiments, the nucleic acid molecule is LNA. In some embodiments, the nucleic acid molecule is a guide RNA (gRNA). In some embodiments, the nucleic acid molecule is a sgRNA. In some embodiments, the pharmaceutical composition further comprises a genome editing enzyme. Genome editing is well known in the art and any such system may be used. In some embodiments, the genome editing enzyme comprises CRISPR/Cas9. In some embodiments, the genome editing enzyme comprises CRISPR/Cas9 or a derivative thereof. In some embodiments, the method comprises reducing FTO expression or function.

In some embodiments, the treating comprises administering an FTO inhibitor. In some embodiments, the treating comprises administering a therapeutically effective amount of an agent. In some embodiments, the treating comprises administering a pharmaceutical composition comprising the agent. In some embodiments, a pharmaceutical composition comprises a pharmaceutically acceptable excipient, adjuvant or carrier.

As used herein, the term “therapeutically effective amount” refers to an amount effective, at dosages and for periods of time necessary, to achieve the desired therapeutic or prophylactic result. The exact dosage form and regimen can be determined by the physician according to the patient's condition.

As used herein, the term “carrier,” “adjuvant” or “excipient” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelies, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein. In some embodiments, a pharmaceutical composition comprises a therapeutically effective amount of the active agent.

The term “FTO inhibitor” refers to any agent which may be a small molecule, an amino acid based molecule, or nucleic acid based molecule, that inhibits the RNA demethylation activity of the FTO enzyme, as identified by Ensembl:ENSG00000140718 MIM:610966; NCBI Reference Sequence: NP_001073901.1, Gene ID: 79068. In some embodiments, the FTO inhibitor is a small molecule. In some embodiments, the FTO inhibitor is an inhibitory compound. In some embodiments, the FTO inhibitor is not a nucleic acid molecule that specifically decreases FTO transcription, translation or both. In some embodiments, the FTO inhibitor is not an inhibitory RNA of FTO. In some embodiments, the FTO inhibitor is not a CRISPR/CAS9 or other genome editing composition for excision or editing of the FTO genetic locus.

In some embodiments, the FTO inhibitor is not a non-steroidal anti-inflammatory drug (NSAID). In some embodiments, the agent is not an NSAID. In some embodiments, the FTO inhibitor is not meclofenamic acid. In some embodiments, the FTO inhibitor is not meclofenamic acid or a derivative thereof. In some embodiments, the FTO inhibitor is not a derivative of meclofenamic acid. In some embodiments, a derivative of meclofenamic acid is selected from mefenamic acid, niflumic acid, and flufenamic acid. In some embodiments, the FTO inhibitor is not an isooxazoline derivative. In some embodiments, the FTO inhibitor is not a derivative of isooxazoline.

In some embodiments, the FTO inhibitor is selected from the group consisting of: Meclofenamic acid, Mefenamic acid, Niflumic acid, Flufenamic acid, 2-(2-toluidino)benzoic acid (2TBA); 2-(3-toluidino)benzoic acid (3TBA); 4-chloro-2-[3-(trifluoromethyl)anilino]benzoic acid (CTB); 5-hydroxydecanoate (5HD) methyl 10,11-dihydro-5H-dibenzo[b,f]azepine-4-carboxylate (MDB), clonixin, CS1, CS2, and 10-hydroxydecanoate (10HD). In some embodiments, the FTO inhibitor is selected from the group consisting of: 2TBA, 3TBA, CTB, 5HD, MDB, clonixin, CS1, CS2 and 10HD. In some embodiments, the FTO inhibitor is selected from the group consisting of: 2TBA, 3TBA, CTB, 5HD, MDB, clonixin, and 10HD. In some embodiments, the FTO inhibitor is selected from the group consisting of: 2TBA, 3TBA, CTB, 5HD, MDB, and 10HD. In some embodiments, the FTO inhibitor is selected from the group consisting of: 2TBA, 3TBA, CTB, 5HD, and MDB. In some embodiments, the FTO inhibitor is selected from the group consisting of: 2TBA, 3TBA, 5HD, and MDB. In some embodiments, the FTO inhibitor is selected from the group consisting of: 2TBA, 3TBA, and MDB. In some embodiments, the FTO inhibitor is selected from the group consisting of: 3TBA, and MDB. In some embodiments, the FTO inhibitor is MDB. In some embodiments, the FTO inhibitor is 3TBA. In some embodiments, the FTO inhibitor is 2TBA. In some embodiments, the FTO inhibitor is 5HD. In some embodiments, the FTO inhibitor is 10HD. In some embodiments, the FTO inhibitor is CTB. In some embodiments, the FTO inhibitor is clonixin. In some embodiments, the FTO inhibitor is mefenamic acid. In some embodiments, the FTO inhibitor is niflumic acid. In some embodiments, the FTO inhibitor is flufenamic acid. In some embodiments, the FTO inhibitor is meclofenamic acid. In some embodiments, the FTO inhibitor is CS1. CS1 is also known as bisantrene (NSC-337766). In some embodiments, the FTO inhibitor is CS2. CS2 is also known as brequinar sodium (NSC-368390).

In some embodiments, the disease is characterized by mRNA instability. In some embodiments, the disease is characterized by nonsense-mediated decay (NMD). In some embodiments, the instability is of an mRNA of a disease-associated gene. In some embodiments, the instability is of a disease-associated mRNA. In some embodiments, the NMD is of an mRNA of a disease-associated gene. In some embodiments, the NMD is of a disease-associated mRNA. In some embodiments, disease-associated is disease-causing. In some embodiments, the mRNA is a disease-associated mRNA. In some embodiments, the mRNA is a disease-causing mRNA. In some embodiments, the mRNA is of a gene. In some embodiments, the gene is a disease-associated gene. In some embodiments, the gene is a disease-causing gene.

As used herein, a “disease-associated gene” is a gene whose function, or loss of function contributes to the disease. In some embodiments, the gene is a protein coding gene. In some embodiments, the gene is not FTO. In some embodiments, decreased expression of the gene is associated with the disease. In some embodiments, decreased expression of the gene causes the disease. In some embodiments, decreased expression is loss of expression. In some embodiments, decreased function of the protein encoded by the gene is associated with the disease. In some embodiments, decreased function of the protein encoded by the gene causes the disease. Examples of genes whose loss/decrease is expression or function are associated with a disease include, but are not limited to, dystrophin in muscular dystrophies, tumor suppressor genes in cancers, splicing genes in cancer, mismatch repair genes in cancer, cystic fibrosis transmembrane conductance regulator (CFTR) in cystic fibrosis, Collagen 6A1-3 (COL6A1, COL6A2, COL6A3) in Ullrich CMD, factor VII in factor VII deficiency, and myophosphorylase (PYGM) in McArdle disease. Methods of determining mRNA instability or NMD in a sample or a subject are well known in the art and any method may be used. Methods of determining mRNA stability or degradation include, but are not limited to, PCR, norther blots, pulse chase experiments, radioactive labeling, reporter assays.

In some embodiments, mRNA instability comprises aberrant mRNA degradation. In some embodiments, mRNA degradation is measured by measuring steady state mRNA levels. In some embodiments, mRNA degradation is measured by measuring mRNA levels. In some embodiments, mRNA degradation is inhibited by cycloheximide. In some embodiments, mRNA instability is confirmed by treatment with cycloheximide and measuring increased mRNA levels.

In some embodiments, the gene comprises a mutation. In some embodiments, mutation creates a premature stop codon. In some embodiments, the mutation creates a premature termination codon (PTC). In some embodiments, the mutation is a loss-of-function mutation. In some embodiments, the mutation increases instability of an mRNA of the gene. In some embodiments, the mRNA comprises a mutation. In some embodiments, the mutation increases degradation of the mRNA. In some embodiments, the PTC causes degradation of the mRNA. In some embodiments, the PCT induces NMD of the mRNA. In some embodiments, the PTC causes decreased levels of the mRNA. In some embodiments, the PTC causes decreased levels of the protein encoded by the mRNA. In some embodiments, the disease is characterized by an mRNA comprising a PTC. In some embodiments, the disease is characterized by the presence of a PTC. In some embodiments, the disease is characterized by an mRNA of the gene that comprises a PTC. In some embodiments, the gene is a disease-associated gene. In some embodiments, the gene is a disease-causing gene.

In some embodiments, the disease is a muscular dystrophy. In some embodiments, the muscular dystrophy is a muscular dystrophy characterized by mRNA instability of a disease-associated gene. In some embodiments, the muscular dystrophy is a muscular dystrophy characterized by NMD of a disease-associated gene. In some embodiments, the muscular dystrophy is Duchenne's muscular dystrophy (DMD). In some embodiments, the muscular dystrophy is Bechet's muscular dystrophy (BMD). In some embodiments, the muscular dystrophy is selected from DMD and BMD. In some embodiments, the muscular dystrophy is oculopharyngeal muscular dystrophy (OPMD). In some embodiments, the muscular dystrophy is selected from DMD, BMD and OPMD. In some embodiments, the disease is a muscular disease, and the gene is a muscle promoting gene. In some embodiments, the disease is a muscular disease. In some embodiments, the muscular disease is a muscular dystrophy. In some embodiments, the muscle promoting gene is Dysrophin.

In some embodiments, the disease is cancer. In some embodiments, the cancer is a cancer characterized by mRNA instability of a disease-associated gene. In some embodiments, the cancer is a cancer characterized by NMD of a disease-associated gene. In some embodiments, the cancer is a solid cancer. In some embodiments, the cancer is a hematological cancer. In some embodiments, the cancer is not characterized by oncogenic FTO. In some embodiments, the cancer is not characterized by oncogenic FTO expression. In some embodiments, the cancer does not comprise oncogenic FTO. In some embodiments, the cancer does not comprise oncogenic FTO expression. In some embodiments, the cancer is lung cancer. In some embodiments, the cancer is acute myeloid leukemia (AML). In some embodiments, the cancer is cryonic myelogenous leukemia (CML). In some embodiments, cancer is selected from lung cancer and AML. In some embodiments, cancer is selected from lung cancer, CIVIL and AML. In some embodiments, the disease is cancer, and the gene is an anti-cancer gene. In some embodiments, an anti-cancer gene is a tumor suppressor gene. In some embodiments, the disease is caner, and the gene is a splicing factor. In some embodiments, the disease is cancer, and the gene is an MMR gene.

In some embodiments, the cancer is a chancer characterized by increased NMD. In some embodiments, the cancer comprises elevated numbers of PTCs. In some embodiments, elevated and increased is as compared to a non-cancerous cell. In some embodiments, a non-cancerous cell is a wild-type cell. In some embodiments, a non-cancerous cell is a healthy cell. In some embodiments, the non-cancerous cell is of the same cell type as the cancerous cell. In some embodiments, a cancer with increased NMD is a cancer with increased aberrant splicing. In some embodiments, a cancer with increased NMD is a cancer with impaired DNA repair. In some embodiments, a cancer with increased NMD is a caner with defective DNA repair. In some embodiments, a cancer with increased NMD is a cancer with increased mutation. In some embodiments, the cancer comprises a mutation of a splicing factor. In some embodiments, a cancer with increased aberrant splicing is a cancer comprising mutation of a splicing factor. In some embodiments, mutation of a splicing factor is mutation of a splicing factor gene. In some embodiments, the cancer comprises aberrant splicing of an mRNA. In some embodiments, the cancer is caused by aberrant splicing of an mRNA. In some embodiments, the cancer is characterized by aberrant splicing of an mRNA. In some embodiments, the cancer is characterized by increased aberrant splicing. In some embodiments, the cancer comprises a mutation of a DNA repair gene. In some embodiments, the cancer is characterized by mutation of a DNA repair gene. DNA repair pathways are well known in the art and include for example the mismatch repair (MMR), the nucleotide excision repair (NER), homologous recombination repair (HRR), non-homologous end joining (NHER) and many others. In some embodiments, the DNA repair is MMR. In some embodiments, the cancer comprises a mutation of a mismatch repair (MMR) gene. In some embodiments, the cancer comprises a mutation in an MMR protein. In some embodiments, the cancer comprises a mutation in a splicing factor gene or an DNA repair gene. In some embodiments, the cancer comprises a mutation in a splicing factor gene or an MMR gene. In some embodiments, the cancer is characterized by a mutation in a splicing factor gene or an DNA repair gene. In some embodiments, the cancer is characterized by a mutation in a splicing factor gene or an MMR gene.

Splicing factors are well known in the art and include, but are not limited to SRSF1, SRSF2, SRSF3, SRSF6, U1, SF3B1, and U2AF1. In some embodiments, the splicing factor is SRSF1. In some embodiments, the splicing factor is SRSF2. In some embodiments, the splicing factor is SRSF3. In some embodiments, the splicing factor is SRSF6. In some embodiments, the splicing factor is U1. In some embodiments, the splicing factor is U2AF1. In some embodiments, the splicing factor is SF3B1. In some embodiments, an aberrantly splicing gene is EZH2. In some embodiments, an aberrantly splicing gene is calpastatin (CAST). In some embodiments, an aberrantly splicing gene is SETX. In some embodiments, an aberrantly splicing gene is TDP52L2. In some embodiments, an aberrantly splicing gene is THYN1.

DNA repair genes are well known in the art and include, but are not limited to, mutS homologs, damage recognition factors, excision factors, ligases, helicases, recombinases, and replication factors to name but a few. MMR genes are also well known in the art and include, but are not limited to, mutS homologs, mutL homologs, exonuclease 1, and replication factors and proteins. In some embodiments, the MMR gene is selected from MSH2, MSH6, MLH1, ERCC1, ERCC4, MBD4, BRCA1, BRCA2, and Rad51. In some embodiments, the MMR gene is MSH6.

In some embodiments, the gene is dystrophin. In some embodiments, the gene is Rev3L. In some embodiments, the gene is MSH6. In some embodiments, the gene is U2AF1. In some embodiments, the gene is SRSF1. In some embodiments, the gene is SRSF3. In some embodiments, the gene is SRSF6. In some embodiments, the gene is ATF3.

In some embodiments, the disease is a genetic disease. In some embodiments, the disease is caused by a point mutation. In some embodiments, the disease is caused by a missense mutation. In some embodiments, the disease is caused by a PTC. In some embodiments, the disease is selected form the group consisting of: muscular dystrophy, cystic fibrosis, Ullrich disease, factor VII deficiency, Hailey-Hailey disease, hemophilia, leucocyte adhesion deficiency 1 (LAD1), cancer, ataxia telangiectasia, Rett syndrome, Usher syndrome type I (USH1), Hurler syndrome (MPS-IH), Maroteaux-Lamy syndrome (MPSVI), carnitine palmitoyltransferase 1A (CPT1A), methylmalonic acidura (MMA), neuronal ceroid lipofuscinosis (NCL), spinal muscular atrophy (SMA), peroxisome biogenesis disorder (PBD), and McArdle disease. In some embodiments, hemophilia is selected from hemophilia A and hemophilia B. In some embodiments, hemophilia is hemophilia A. In some embodiments, hemophilia is hemophilia B. In some embodiments, the disease is an obesity related disorder. In some embodiments, the disease is a pathological condition related to bone-mineral density. In some embodiments, the obesity disorder is related to bone-mineral density. In some embodiments, related to bone-mineral density is bone-mineral density disorder.

In some embodiments, the method further comprises confirming mRNA instability of the mRNA before said administering. In some embodiments, the method further comprises confirming mRNA instability in the subject before the administering. In some embodiments, the method further comprises confirming NMD of the mRNA before said administering. In some embodiments, the method further comprises confirming NMD in the subject before the administering. In some embodiments, the confirming comprises receiving a sample from the subject and confirming within the sample. In some embodiments, the sample is a bodily fluid. In some embodiments, the sample is a biopsy. In some embodiments, the sample is a disease sample. In some embodiments, the sample is from a diseased tissue. A skilled artisan will appreciate that many diseases act locally, and the sample may be from a diseased location. For example, a muscle sample may be analyzed when the disease is a muscular dystrophy. In some embodiments, the sample is a muscle sample. In some embodiments, the sample is a sample comprising cells that express the gene. In some embodiments, the sample is a sample comprising cells that when healthy express the gene. In some embodiments, a bodily fluid is selected from blood, serum, gastric fluid, intestinal fluid, saliva, bile, tumor fluid, breast milk, urine, interstitial fluid, cerebral spinal fluid and stool. In some embodiments, the bodily fluid is blood. In some embodiments, the bodily fluid is serum. In some embodiments, the confirming comprises extracting mRNA from the sample. In some embodiments, the confirming is in the mRNA.

In some embodiments, the method further comprises administering at least one read-through promoting agent. As used herein, the term “read-through promoting agent” refers to any drug or compound that increases or promotes continued translation through a premature termination codon. The readthrough promoting agent is a compound, which may be a small molecule, amino acid based molecule, nucleic acid based molecule, that enables translation from a mRNA transcript while disregarding the presence of a stop codon in the mRNA transcript. This can be achieved, for example, by binding to either the 40S or 60S subunit of the ribosome and decrease the fidelity of the stop codon. Read-through promoting agents are well known in the art and any such agent may be used. In some embodiments, the read-through promoting agent is a small nucleic acid molecule. In some embodiments, the read-through promoting agent is an antisense oligonucleotide (ASO). In some embodiments, the read-through promoting agent is a drug. In some embodiments, the administering comprises administering a pharmaceutical composition comprising the read-through promoting agent.

In some embodiments, the read-through promoting agent is selected from the group consisting of: aminoglycosides, modified aminoglycosides, erythromycin, azithromycin, (5Z)-2-Amino-5-[[5-(2-nitrophenyl)-2-furanyl]methylene]-4(5H)-thiazolone (RTC13), 3-[5-(2-Fluorophenyl)-1,2,4-oxadiazol-3-yl]benzoic acid (Ataluren), and 2-amino-7-isopropyl-5-oxo-5H-chromeno[2,3-b]pyridine-3-carboxylic acid (Amlexanox). In some embodiments, the read-through promoting agent is erythromycin. In some embodiments, the read-through promoting agent is azithromycin. In some embodiments, the read-through promoting agent is RTC13. In some embodiments, the read-through promoting agent is Ataluren. In some embodiments, the read-through promoting agent is Amlexanox.

By another aspect, there is provided a pharmaceutical composition comprising at least one FTO inhibitor and at least one read-through promoting agent. By another aspect, there is provided a pharmaceutical composition comprising at least one agent that decreases FTO expression or function and at least one read-through promoting agent.

By another aspect, there is provided a kit comprising at least one agent that decreases FTO expression or function and at least one read-through promoting agent. By another aspect, there is provided a kit comprising at least one FTO inhibitor and at least one read-through promoting agent.

In some embodiments, the kit comprises a pharmaceutical composition comprising the at least one FTO inhibitor. In some embodiments, the kit comprises a pharmaceutical composition comprising the at least one read-through promoting agent. In some embodiments, the kit comprises s a label stating the FTO inhibitor and the read-through promoting agent are for use in combination. In some embodiments, the kit is for use in combination therapy to treat a disease.

The FTO inhibitor and the read-through promoting agent may be administered simultaneously or separately. The agents may be administered in the same pharmaceutical composition, using the same pharmaceutically acceptable carrier, or in two different compositions, each having its own acceptable carrier.

As used herein, the terms “administering,” “administration,” and like terms refer to any method which, in sound medical practice, delivers a composition containing an active agent to a subject in such a manner as to provide a therapeutic effect. One aspect of the present subject matter provides for oral administration of a therapeutically effective amount of a composition of the present subject matter to a patient in need thereof. Other suitable routes of administration can include parenteral, subcutaneous (SC), intravenous (IV), intramuscular, or intraperitoneal. The FTO inhibitor and the read-through promoting agent may be administered by the same mode of administration or by two different modes of administration, for example one orally and the other one by IV/SC injection. The two active agents may be administered using the same administration protocol (for example: one, twice or three times daily) or different administration protocols (for example: one given twice daily and the other given once daily/twice weekly etc.). In some embodiments, the pharmaceutical composition is formulated for systemic administration. In some embodiments, the pharmaceutical composition is formulated for oral administration. In some embodiments, the pharmaceutical composition is formulated for intramuscular administration. In some embodiments, the pharmaceutical composition is formulated for intravenous administration.

The dosage administered will be dependent upon the age, health, and weight of the recipient, kind of concurrent treatment, if any, frequency of treatment, and the nature of the effect desired.

By another aspect, there is provided an agent that decreases FTO expression or function for use in treating a disease in a subject in need thereof. By another aspect, there is provided an FTO inhibitor for use in treating a disease in a subject in need thereof. By another aspect, there is provided a pharmaceutical composition comprising an agent that decreases FTO expression or function for use in treating a disease. By another aspect, there is provided a pharmaceutical composition comprising an FTO inhibitor for use in treating a disease. By another aspect, there is provided a pharmaceutical composition comprising an agent that decreases FTO expression or function and a read-through promoting agent for use in treating a disease. By another aspect, there is provided a pharmaceutical composition comprising an FTO inhibitor and a read-through promoting agent for use in treating a disease. By another aspect, there is provided a kit comprising an agent that decreases FTO expression or function and a read-through promoting agent for use in treating a disease. By another aspect, there is provided a kit comprising an FTO inhibitor and a read-through promoting agent for use in treating a disease.

By another aspect, there is provided a method of determining suitability of a subject suffering from a disease to be treated with an agent that decreases FTO expression or function, the method comprising measuring mRNA stability in the subject, wherein determining instability of mRNA indicates the subject is suitable for treatment with an agent.

By another aspect, there is provided a method of determining suitability of a subject suffering from a disease to be treated with an FTO inhibitor, the method comprising measuring mRNA stability in the subject, wherein determining instability of mRNA indicates the subject is suitable for treatment with an FTO inhibitor.

In some embodiments, the method comprises measuring mRNA stability of an mRNA of a gene. In some embodiments, the gene is a gene associated with the disease. In some embodiments, the gene is a gene that causes the disease. In some embodiments, measuring mRNA stability is measuring NMD. In some embodiments, detecting NMD indicates the subject is suitable for treatment. In some embodiments, detecting is detecting above a predetermined threshold. In some embodiments, detecting is detecting above levels present in a healthy subject.

In some embodiments, the method comprises receiving a sample from the subject. In some embodiments, the measuring is measuring in the sample. In some embodiments, the measuring is measuring mRNA stability in the sample. In some embodiments, the sample is a sample comprising cells that express the gene. In some embodiments, the sample is a sample comprising cells that when healthy express the gene. In some embodiments, the sample is a sample of a tissue that can be afflicted with the disease.

By another aspect, there is provided a method of inhibiting FTO, the method comprising contacting the FTO with a small molecule selected from the group consisting of: 2-(2-toluidino)benzoic acid (2TBA); 2-(3-toluidino)benzoic acid (3TBA); 4-chloro-2-[3-(trifluoromethyl)anilino]benzoic acid (CTB); 5H-Dibenz[b,f]azepine (5HD), Clonixin, 10H-Dibenz[b,f]azepine (10HD), and methyl 10,11-dihydro-5H-dibenzo[b,f]azepine-4-carboxylate (MDB).

By another aspect, there is provide a small molecule selected from the group consisting of: 2-(2-toluidino)benzoic acid (2TBA); 2-(3-toluidino)benzoic acid (3TBA); 4-chloro-2-[3-(trifluoromethyl)anilino]benzoic acid (CTB); 5H-Dibenz[b,f]azepine (5HD), Clonixin, 10H-Dibenz[b,f]azepine (10HD), and methyl 10,11-dihydro-5H-dibenzo[b,f]azepine-4-carboxylate (MDB) for use in inhibiting FTO.

In some embodiments, the contacting is with 2TBA. In some embodiments, the contacting is with 3TBA. In some embodiments, the contacting is with CTB. In some embodiments, the contacting is with 5HD. In some embodiments, the contacting is with 10HD. In some embodiments, the contacting is with MDB. In some embodiments, the 2TBA is for use in inhibiting FTO. In some embodiments, the 3TBA is for use in inhibiting FTO. In some embodiments, the CTB is for use in inhibiting FTO. In some embodiments, the 5HD is for use in inhibiting FTO. In some embodiments, the 10HD is for use in inhibiting FTO. In some embodiments, the MDB is for use in inhibiting FTO. In some embodiments, the contacting is with a pharmaceutical composition comprising the small molecule. In some embodiments, a pharmaceutical composition comprising the small molecule is for use in inhibiting FTO.

In some the inhibiting is inhibiting FTO in a cell. In some embodiments, the inhibiting is inhibiting FTO in a subject. In some embodiments, the subject is a subject in need thereof. In some embodiments, the subject is a subject suffering from a condition treatable by FTO inhibition. In some embodiments, the disease is a disease treatable by FTO inhibition. In some embodiments, the small molecule is an FTO inhibitor.

As used herein, the term “inhibiting FTO” and “FTO inhibition” are used interchangeably and refer to decreasing the function of FTO. In some embodiments, inhibiting FTO does not comprise reducing the expression of FTO. In some embodiments, inhibiting FTO does not comprise degrading FTO. In some embodiments, inhibiting FTO does not comprise reducing the amount of FTO present. In some embodiments, present is present in the cell. In some embodiments, present is present in the subject. In some embodiments, FTO function is demethylation of m6A. In some embodiments, FTO function is catalyzing demethylation of m6A. In some embodiments, the demethylation is oxidative demethylation. In some embodiments, m6A is methylation of adenosine 6. In some embodiments, m6A is N6-methyladenosine. In some embodiments, the m6A is on an RNA. In some embodiments, the RNA is an mRNA. In some embodiments, inhibiting of FTO is inhibition of demethylation of m6A by FTO. In some embodiments, the inhibition is at least 50% inhibition. In some embodiments, the inhibition is at least 55% inhibition. In some embodiments, the inhibition is at least 60% inhibition. In some embodiments, the inhibition is at least 65% inhibition. In some embodiments, the inhibition is at least 70% inhibition. In some embodiments, the inhibition is at least 75% inhibition. In some embodiments, the inhibition is at least 80% inhibition. In some embodiments, the inhibition is at least 85% inhibition. In some embodiments, the inhibition is at least 90% inhibition. In some embodiments, the inhibition is at least 95% inhibition. In some embodiments, the inhibition is at least 97% inhibition. In some embodiments, the inhibition is at least 99% inhibition. In some embodiments, the inhibition is 100% inhibition.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

TABLE 1
Compounds

| Reagent | Source | Cat. no. |
|---|---|---|
| Cyclohexamide | Cell signaling | 2112 |
| 5-azacytidine | Abcam | ab142744 |
| Amlexanox | Abcam | ab142825 |
| PTC124 | ENCO | A10758 |
| Meclofenamic acid | Sigma | M4531 |
| Mefenamic acid | Sigma | M4267 |
| Flufenamic acid | Sigma | F9005 |
| Niflumic acid | Sigma | N0630 |
| 2-(2-toluidino)benzoic acid (2TBA) | Sigma | PH004265 |
| 2-(3-toluidino)benzoic acid (3TBA) | Sigma | PH009774 |
| 4-chloro-2-[3-(trifluoromethyl)anilino] | Sigma | PH002043 |
| benzoic acid (CTB) | | |
| methyl 10,11-dihydro-5H- | Sigma | PH006463 |
| dibenzo[b,f]azepine-4-carboxylate (MDB) | | |
| Clonixin (Clo) | Sigma | SML0530 |
| Flunixin Meglumine (Flun) | Sigma | PHR1442 |
| 5H-Dibenz[b,f]azepine (5HD) | Sigma | 143650 |
| 10,11-Dihydro-5H- | Sigma | I1308 |
| dibenz[b,f]azepine (10HD) | | |
| Entacapone (Ent) | Sigma | SML0654 |
| Azithromycin (Azi) | Sigma | PHR1088 |
| Erythromycin (Ery) | Sigma | E5389 |
| Carbazole (Car) | Sigma | C5132 |
| Cisplatin (CDDP) | Sigma | C2210000 |
| Matrigel high concentration | Corning | 354248 |
| Doxycycline hyclate | Sigma | D9891 |

TABLE 2
Antibodies

| Reagent | Source | Cat. No. |
|---|---|---|
| Anti- | Abcam | Ab15277 |
| Dystrophin | | |

| | |
|---|---|
| Anti- | mAb AK96 culture supernatant |
| SRSF1 | |
| Anti- | mAb 8-1-28 culture supernatant |
| SRSF6 | |

| | | |
|---|---|---|
| Anti- | Sigma | HPA043484 |
| SRSF5 | | |
| Anti-β- | Sigma | C7207 |
| catenin | | |
| Anti-β- | Santa cruz | sc-1616 |
| Actin | | |
| Anti-β- | Sigma | T8535 |
| Tubulin | | |
| I + II | | |

| | |
|---|---|
| Anti-UPF1 | Gifted |

| | | |
|---|---|---|
| Anti-FTO | Abcam | ab 92821 |
| Anti- | Abcam | ab195352 |
| METTL3 | | |
| Anti- | Sigma | HPA007196 |
| ALKBH5 | | |
| Anti- | Sigma | G9545 |
| GAPDH | | |
| Anti- | Santa cruz | sc-47740 |
| MBNL | | |
| Anti- | BD Transduction Laboratories | 610919 |
| MSH6 | | |

TABLE 3
Primers

| Gene | Forward (SEQ ID NO:) | Reverse (SEQ ID NO:) |
|---|---|---|
| e3-e4 | CAGCATATTGAGAACCTCTTC (1) | CTGCAAAACCCGCAGTGCC (2) |
| e65-e66 | CTGGCTGCTGAATGTTTATGATA (3) | CTTGCCACTTGCTTGAAAAG (4) |
| (DP71) | | |
| ATF3 | GCCATTGGAGAGCTGTCTTC (5) | GGGCCATCTGGAACATAAGA (6) |
| RPL3 | GGCATTGTGGGCTACGTG (7) | CTTCAGGAGCAGAGCAGA (8) |
| GAPDH | TGAGCTTGACAAAGTGGTCG (9) | GGCTCTCCAGAACATCATCC (10) |
| Dp140 | GCTTGAGTCATGGAAGGAGGG (11) | GGAGGTCTTTGGCCAACTG (12) |
| e78-e79 | CTTCTCAGTCCTCCCCAGGACAC (13) | CTTGTAAACTCTTACTGTCTAATCC (14) |
| Creatine | GGGCTACAAACCCACTGACA (15) | AACGTGTAGCCCTTGATGCT (16) |
| Kinase | | |
| Troponin T | AGCGGAAGAAGGAGGAAGAG (17) | GTTCGCGTTCCTTCTCAGTT (18) |
| Desmin | AGCGCAGAATTGAATCTCTCA (19) | ACCTGCTGTTCCTGAAGCTG (20) |
| Myogenin | GGTGCCCAGCGAATGC (21) | TGATGCTGTCCACGATGGA (22) |
| FTO | CACCGCTGATCAGAAGCCAGAATGT (23) | AAACACATTCTGGCTTCTGATCAGC (24) |
| guide KO | | |
| crispr | | |
| METTL3 | CACCGGAGTTGATTGAGGTAAAGCG (25) | AAACCGCTTTACCTCAATCAACTCC (26) |
| guide KO | | |
| crispr | | |
| ALKBH5 | CACCGCATCAGGGTCTGCCTTGCGG (27) | AAACCCGCAAGGCAGACCCTGATGC (28) |
| guide KO | | |
| crispr | | |
| o/e FTO | GGGGAAGAATTCACCAGCCACCATGGATGA | TTCCCCGTCGACTCAAGCGTAATCTGGAACATC |
| cloning | AGCGCACCCCGACTGC (29) | GTATGGGTAGGGTTTTGCTTCCAGAA (30) |
| o/e | GGGGAAGAATTCACCAGCCACCATGGATGG | TTCCCCGTCGACTCAAGCGTAATCTGGAACATC |
| ALKBH5 | CGGCCGCCAGCGGCTA (31) | GTATGGGTAAAATATATTAGATTTGGTTT |
| cloning | | (32) |
| o/e | GGGGAATACGTAACCAGCCACCATGTACCC | GGGGAAGTCGACCTATAAATTCTTAGGTTTAGA |
| METTL3 | ATACGATGTTCCAGATTACGCTTCGGACAC | G (34) |
| cloning | GTGGAGCTCTAT (33) | |

Cells: Primary skin fibroblasts were collected from patients in Hadassah Medical Center with approval of the Ethics Committee (Helsinki approval) of the Hebrew University—Hadassah Medical Center, Primary skin fibroblasts were cultured in EMEM media supplemented with 10% of (v/v) fetal calf serum (FBS), penicillin and streptomycin. HeLa, HEK293 and Ovca 433 cells were cultured in DMEM media supplemented with 10% of (v/v) fetal calf serum (FBS), penicillin and streptomycin. NCI-727, Kasumil, NKM and K562 cells were cultured in RPMI media supplemented with 10% of (v/v) fetal calf serum (FBS), penicillin and streptomycin.

MyoD trans-differentiation: Primary skin fibroblasts were seeded in 10 cm plates and grown to 70% confluence. 24 hours later cells were infected for 24 h with MyoD inducible viral system (3×Flag-tagged full-length human MYOD1 cDNA-T2A-dsRed-Express2 cassette expressed from the Tetracycline Responsive Element (TRE) promoter. T2A is a peptide that facilitates ribosomal skipping as the mRNA transcript is being translated into protein). 48 hours after infection, cells were seeded in 6-well plates treated with Matrigel. MyoD inducible system transgene expression was induced by supplementing the medium with 3 μg/ml doxycycline. Fresh media with doxycycline was supplemented every 2 days. All differentiation studies were conducted in standard growth medium. Cells were harvested after 11 days of doxycycline treatment.

Q-RT-PCR: RNA was isolated using. Tri-reagent (Sigma). cDNA synthesis was performed using the High Capacity cDNA Reverse Transcription Kit (Applied Biosystems). Q-RI-PCR was performed using Fast SYBR Green Master Mix (Applied Biosystems) and the StepOnePlus System (Applied Biosystems).

Western blot analysis: Cells were lysed in Laemmli buffer (10% glycerol, 0.05M Tris pH 6.8, 5% β-mercaptoethanol, 3% SDS) and analyzed for total protein concentration. A total of 30 μg of total protein from each cell lysate was separated by SDS-PAGE and transferred onto a PVDF membrane. The membranes were blocked, probed with antibodies, and signal was detected using enhanced chemiluminescence detection. Primary antibodies used are listed in the table.

Crispr-Cas9 viruses: The target gRNA oligonucleotides (listed in the table) were subcloned into the pLenti CRISPR V2 plasmid. Cells were transduced with lentivirus. After infection the cells underwent selection using puromycin (2 μg/μl) for 96 hours.

Trypan-blue exclusion assay: Cells (2×10⁶) were seeded in 6-well plates and further treated with inhibitors at different concentrations (as described in figure). After 48 hours cells were trypsinized and collected, including cells in the medium and PBS wash. Cells were resuspended in HBSS and the percentage of dead cells was determined by 0.4% trypan blue staining using a BioRad cell counter.

Treatment with compounds: 24 hours after cells were seeded the medium was replaced to medium containing the compounds for the indicated amount of time as described in the relevant figure legends. The cells were harvested for either RNA analysis using TRI Reagent (Sigma) or western blot analysis using Laemmli buffer.

### EXAMPLES

### Example 1: Genetic Disruption of FTO

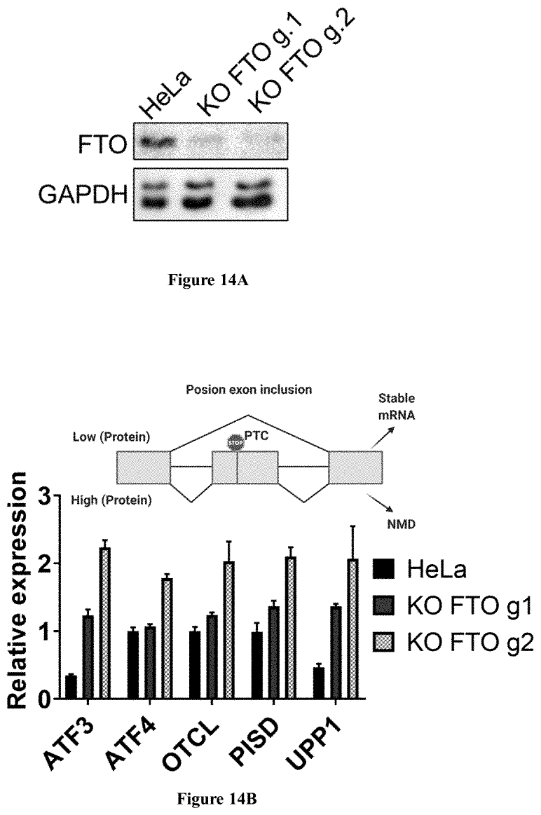

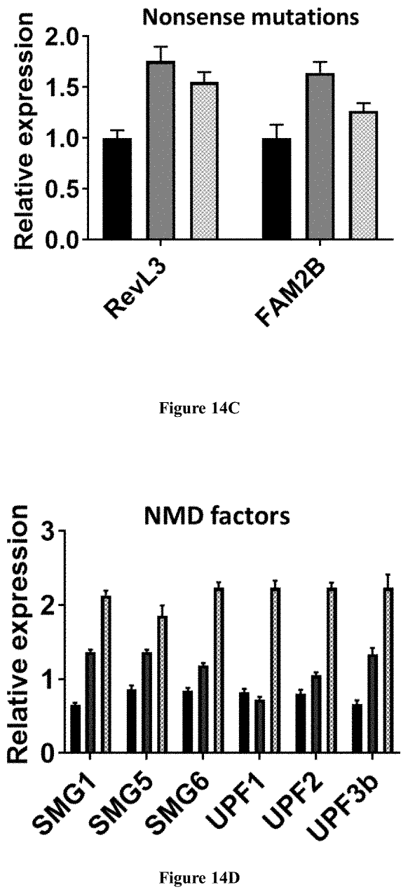

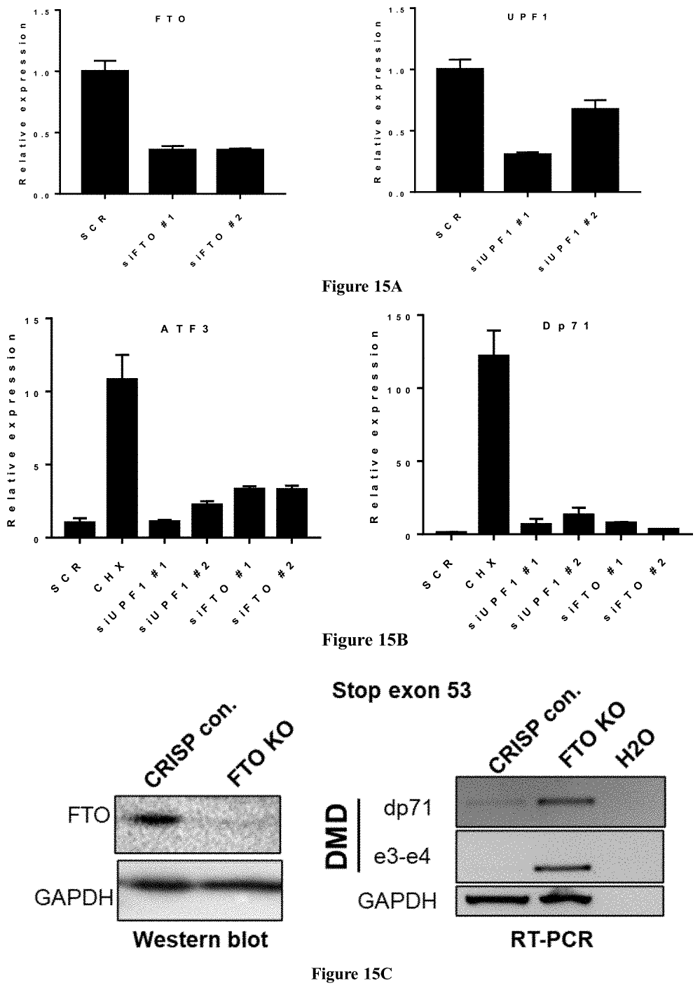

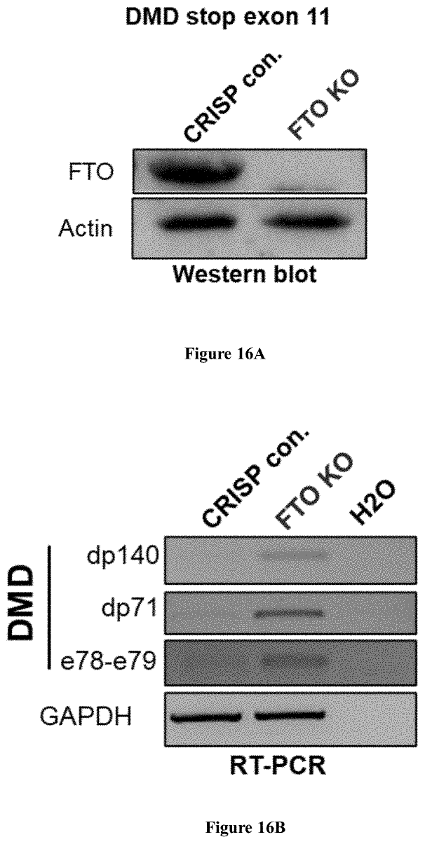

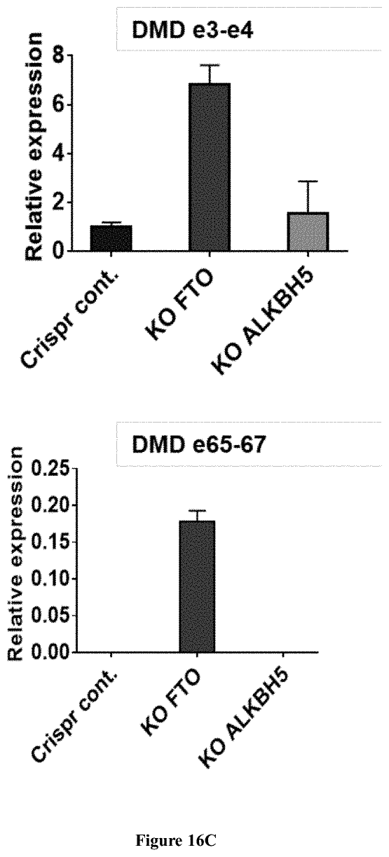

In order to determine if genetic manipulation of enzymes that modulate m⁶A methylation affect NMD and specifically the levels of dystrophin mRNA in Duchenne's muscular dystrophy (DMD) patients, patient-derived fibroblasts were tested. Specifically, primary skin fibroblasts of a DMD patient with a nonsense mutation in exon 53 of the dystrophin gene were transfected with an siRNA that targets FTO (FIG. 15A). As a positive control, cells were transfected with an siRNA that targets UPF1, an essential component of the NMD pathway (FIG. 15A). Although fibroblasts do not express dystrophin protein, they do express the mRNA and thus can be used to test effects on NMD. Knockdown of FTO with siRNA resulted in increased mRNA expression of dystrophin and ATF3 mRNA (FIG. 15B). An alternative to siRNA knockdown is the use of the CRISPR/Cas9 system to induce knockout of the FTO gene using FTO specific guide RNAs (FIG. 14A). HeLa cells knocked out for FTO showed increased mRNA levels of NMD-prone targets (FIG. 14B), two genes that are known to have a nonsense mutation in HeLa cells (FIG. 14C) and NMD core factors (FIG. 14D). Similarly, knockout of FTO in DMD patient-derived fibroblasts containing a nonsense mutation in exon 53 resulted in increased levels of dystrophin mRNA (FIG. 15C). Knockout of FTO (FIG. 16A) in DMD patient-derived fibroblasts containing a nonsense mutation in exon 11 also resulted in increased levels of dystrophin mRNA (FIG. 16B-C).

### Example 2: Pharmacological Inhibition of NMD and FTO

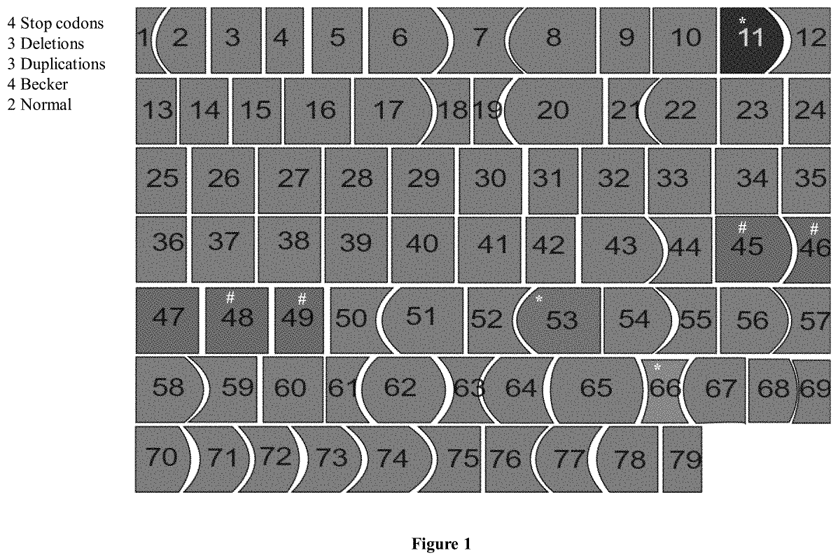

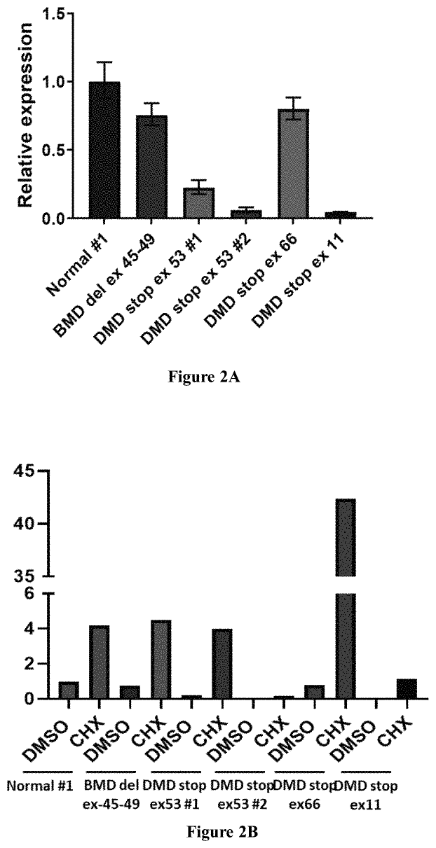

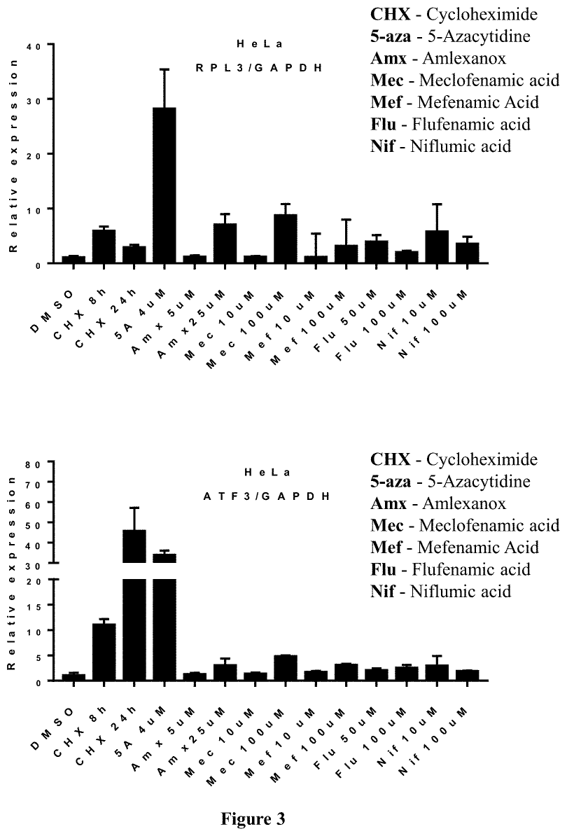

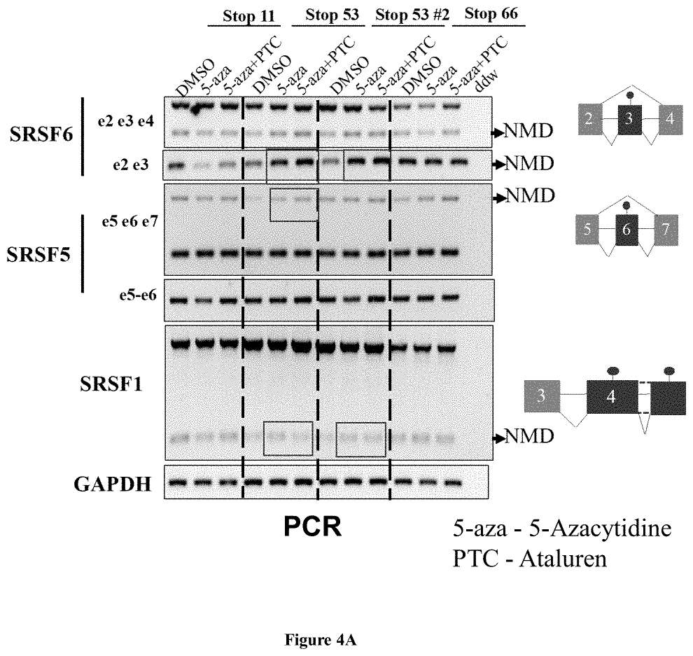

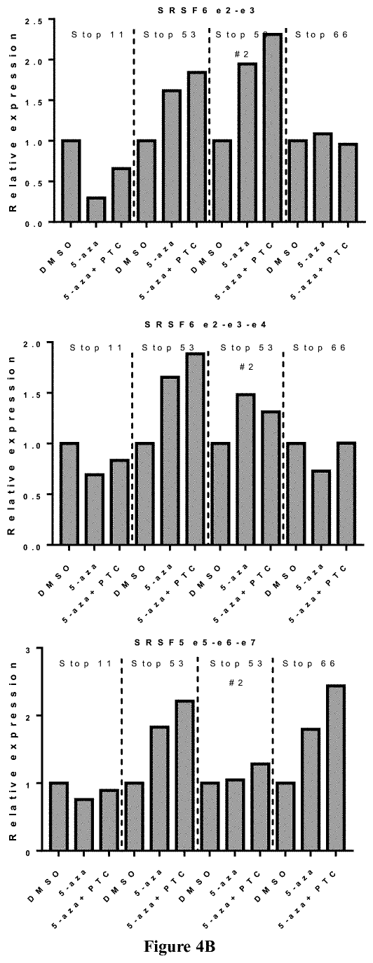

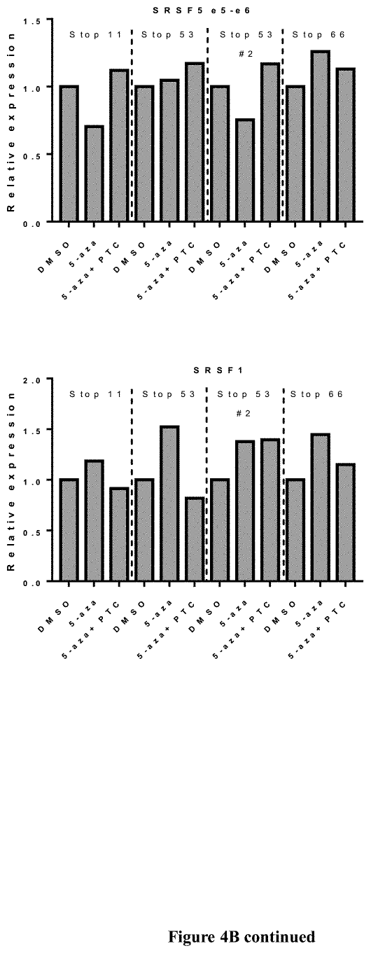

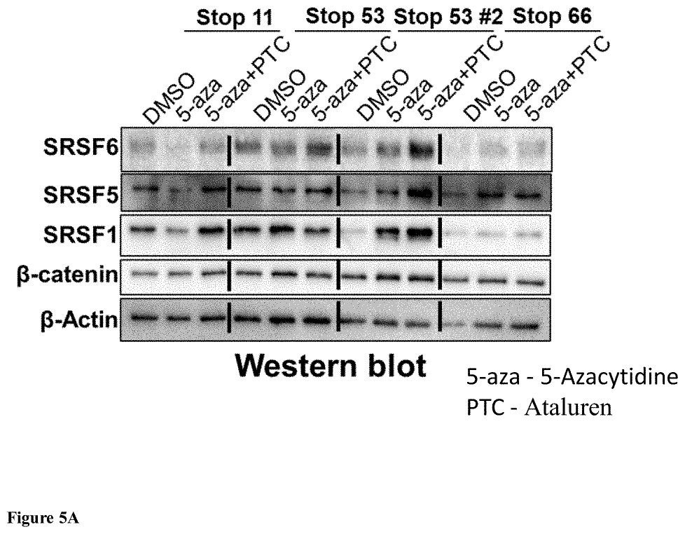

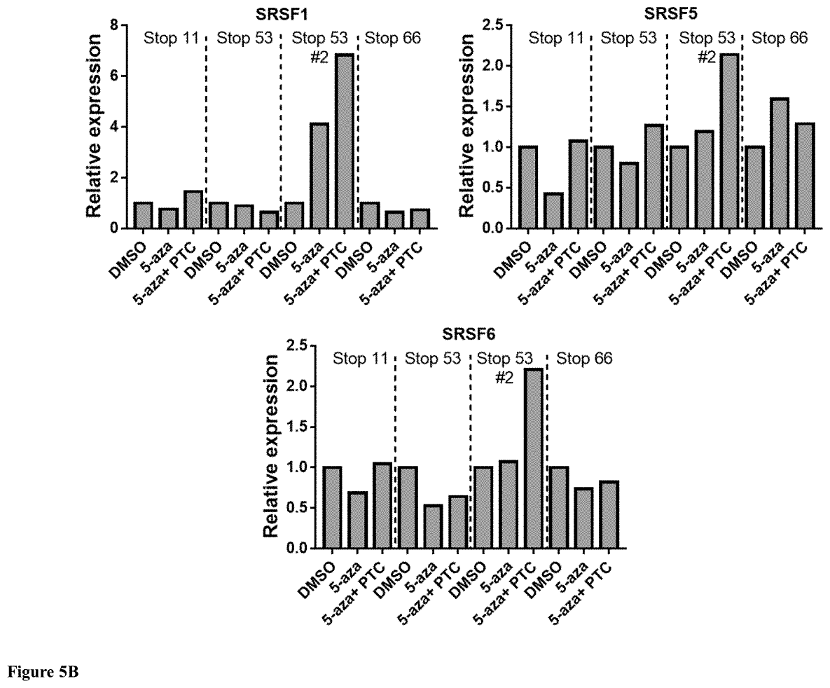

It was hypothesized that dystrophin nonsense-containing mRNAs are unstable compared to wild-type dystrophin mRNA. Skin fibroblasts from 4 DMD patients harboring nonsense mutations, 3 DMD patients with deletions, 3 with duplications, 4 BMD patients with in-frame mutations and 2 normal males were collected (FIG. 1). Treatment of these patient-derived cells with cycloheximide showed stabilization of dystrophin mRNA for many of the subjects (FIG. 2A-B). The fact that dystrophin mRNA can be stabilized by cycloheximide suggests that this mRNA is unstable and is degraded by the NMD pathway. Next, known NMD inhibitors were analyzed to see if they can stabilize dystrophin mRNA in patient-derived fibroblasts. 5′-AzaC and Amlexanox, two known NMD inhibitors, were examined. It was found that these drugs stabilize endogenous NMD-prone mRNAs, ATF3 and RPL3, in HeLa cells (FIG. 3). Because dystrophin protein is only expressed in muscle cells, other ubiquitously expressed proteins which can undergo NMD at the RNA level where tested in fibroblasts. Thus, proteins from the SR protein family were examined, since many of these genes auto-regulated their own expression by alternative splicing-coupled NMD. Treatment of patient-derived fibroblasts from 4 DMD patients with 5′-AzaC alone or in combination with PTC-124 (read-through reagent) resulted in stabilization of SRSF1, SRSF5 and SRSF6 mRNA (FIG. 4A-B) and increased protein production in several cases (FIG. 5A-B).

m⁶A RNA methylation was implicated in several RNA processing steps, including mRNA stability, translation and splicing. However, the connection of NMD to m⁶A methylation is unknown. FTO is a known m⁶A mRNA methylation eraser. FTO inhibitor (Meclofenamic acid) or its analogs (Mefenamic acid, Flufenamic acid and Niflumic acid) were tested as to whether they have an effect on stabilization of NMD-prone mRNAs. Treatment of HeLa cells with an FTO inhibitor or its analogs resulted in stabilization of endogenous NMD-prone mRNAs, ATF3 and RPL3 (FIG. 3).

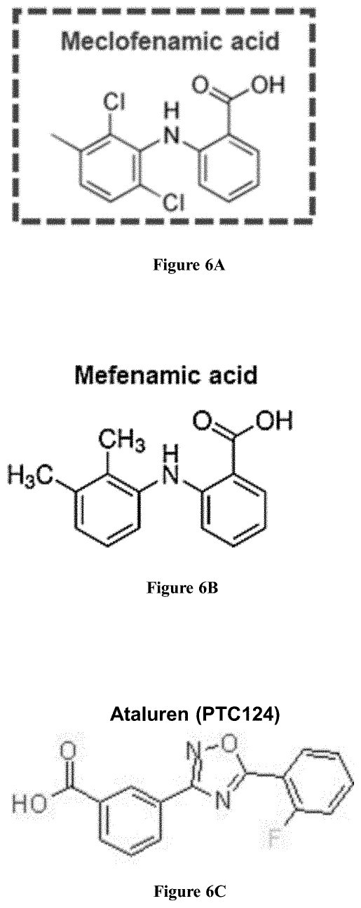

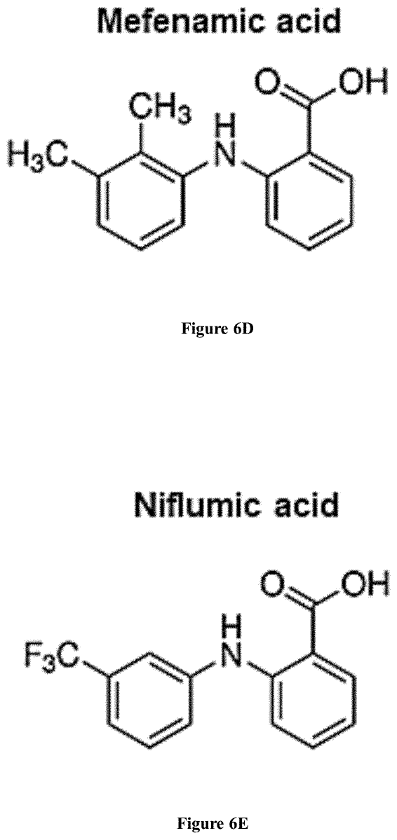

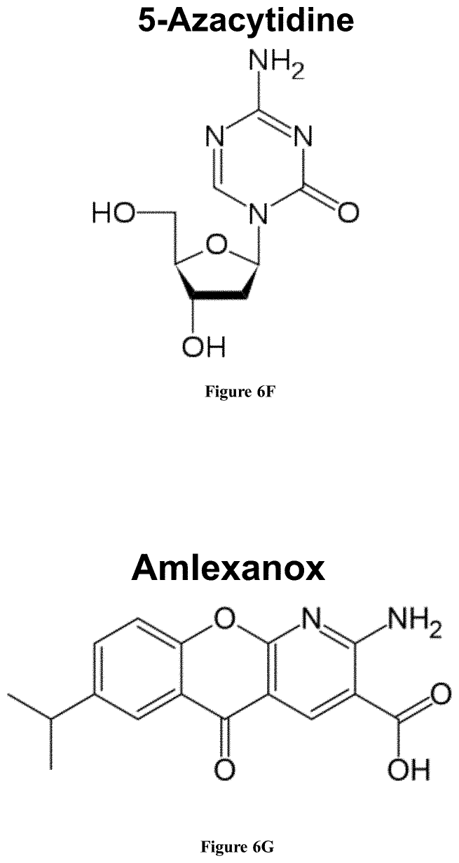

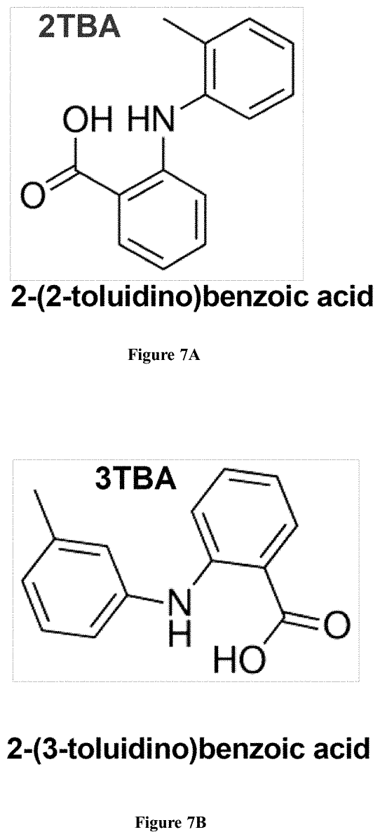

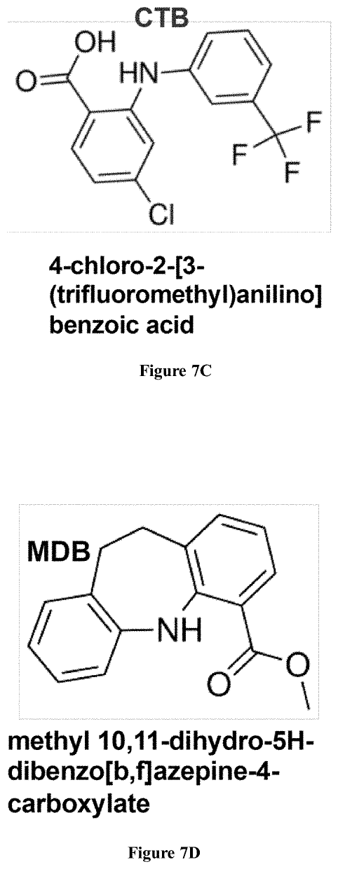

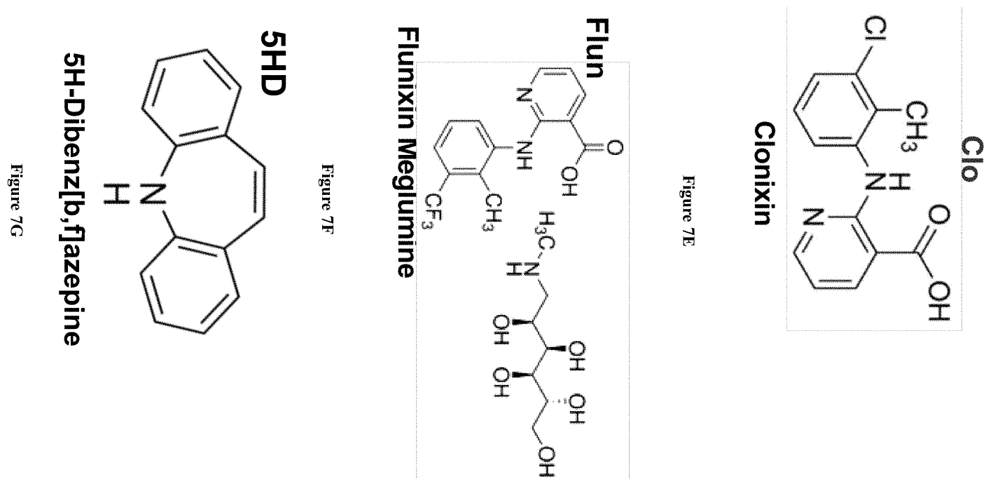

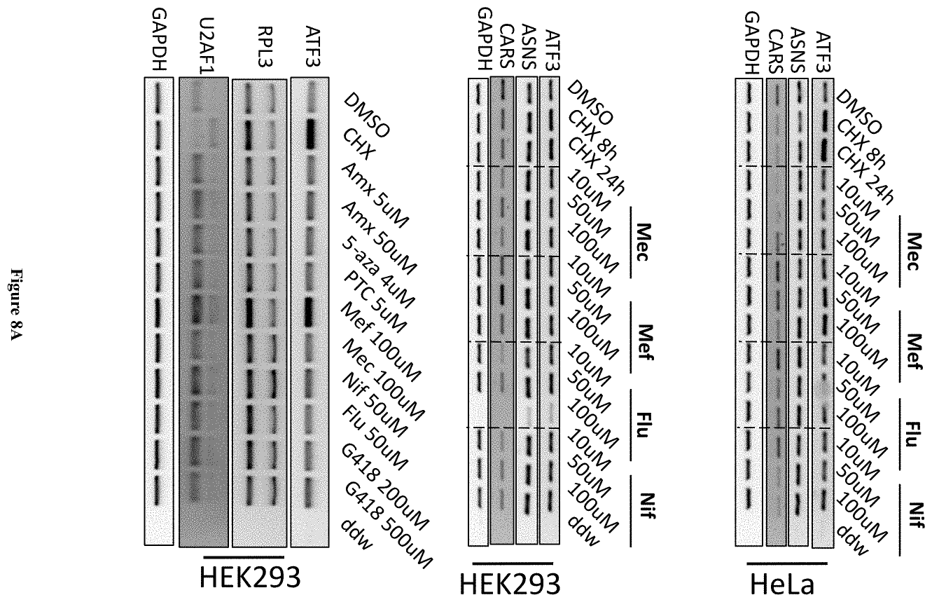

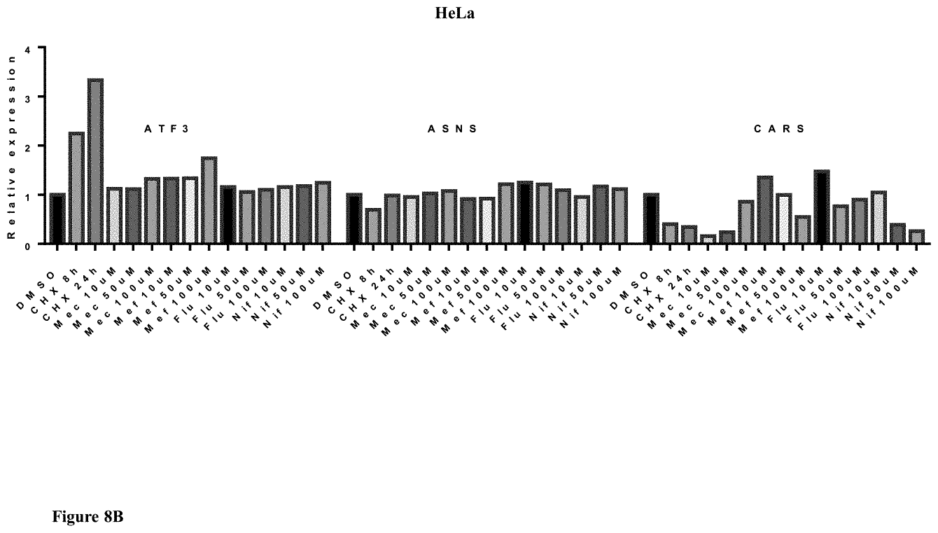

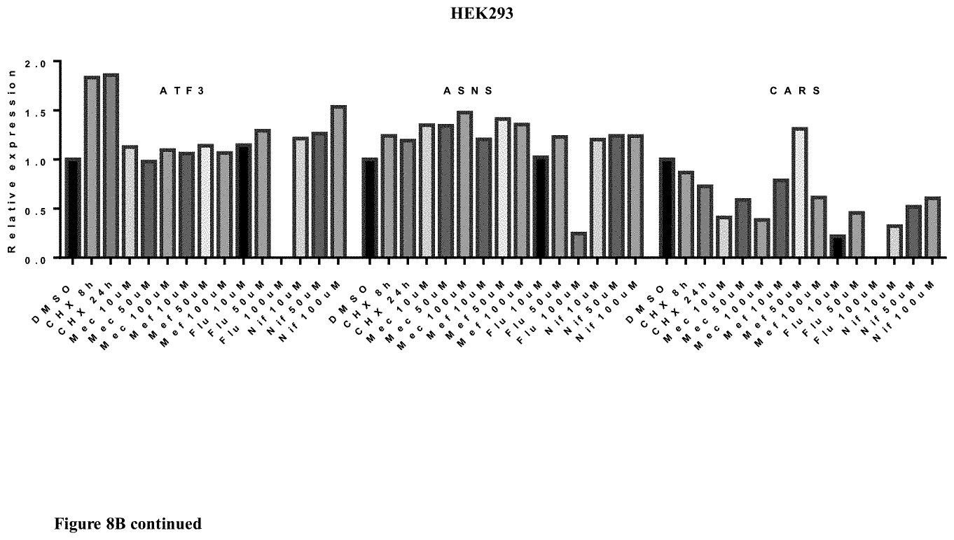

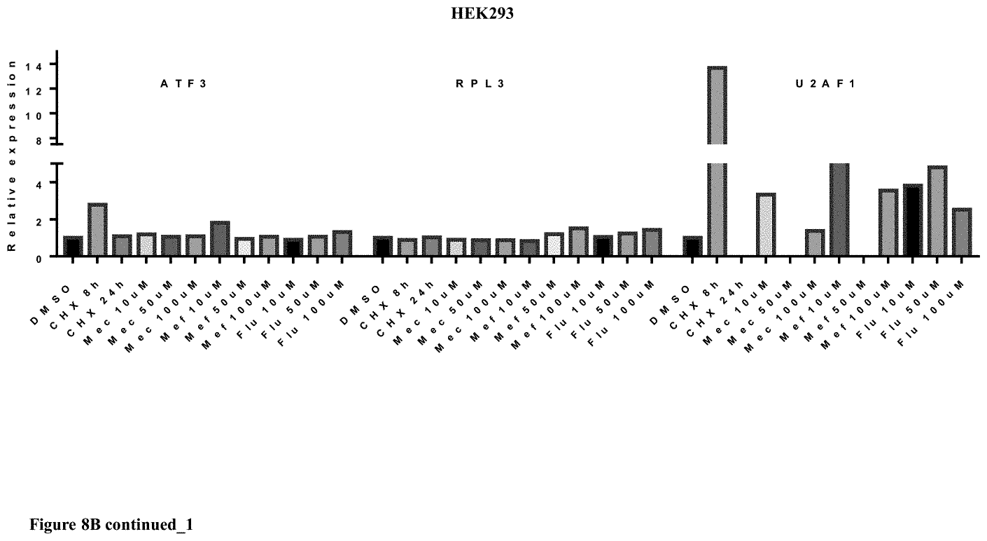

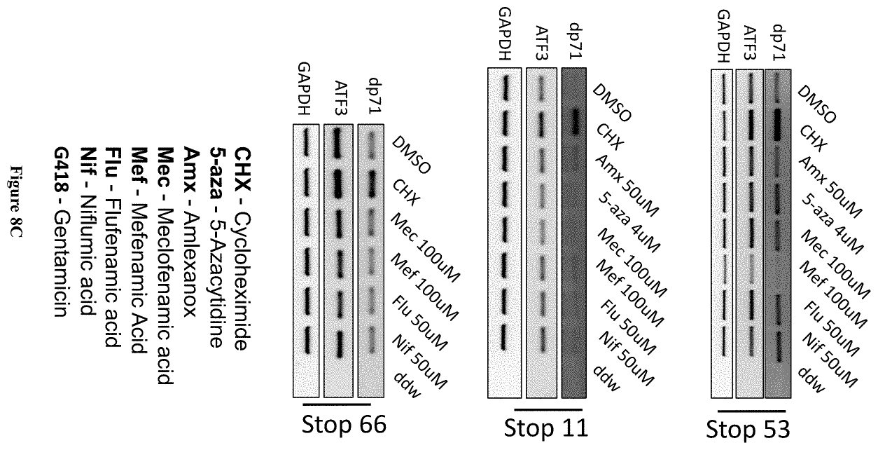

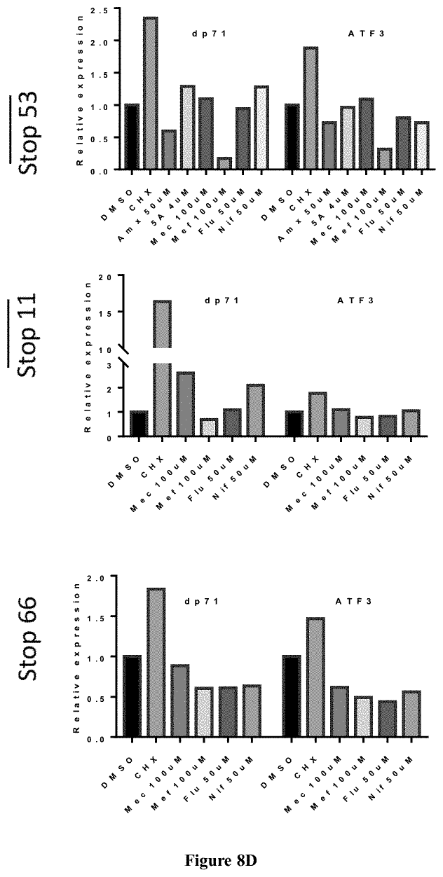

FIGS. 6A-G provide the chemical structure of the above tested molecules. Several novel small molecules that inhibit FTO were also identified (FIG. 7A-G). Initially the first set of compounds was tested for their effect on the stability of NMD-prone transcripts in HeLa and HEK293 cells. Increased expression of NMD-prone transcripts was observed after treatment of these cells with these compounds (FIG. 8A-B). In particular, meclofenamic acid (Mec) and three of its derivatives, mefenamic acid (Mef), flufenamic acid (Flu) and niflumic acid (Nif) all show at least some increase in expression, though some compounds appeared to have more effect on specific genes than others. Nest, three sets of DMD patient-derived cells with different nonsense mutations were tested for the effect of these compounds on dystrophin expression. Once again, all the tested compounds produced increased levels of dystrophin mRNA after treatment though the results were again variable between subjects and compounds (FIG. 8C-D). These results suggest that drugs, which inhibit FTO, have an effect on the stability of NMD-prone transcripts and DMD mRNAs in HeLa, HEK293 and DMD patient-derived cells.

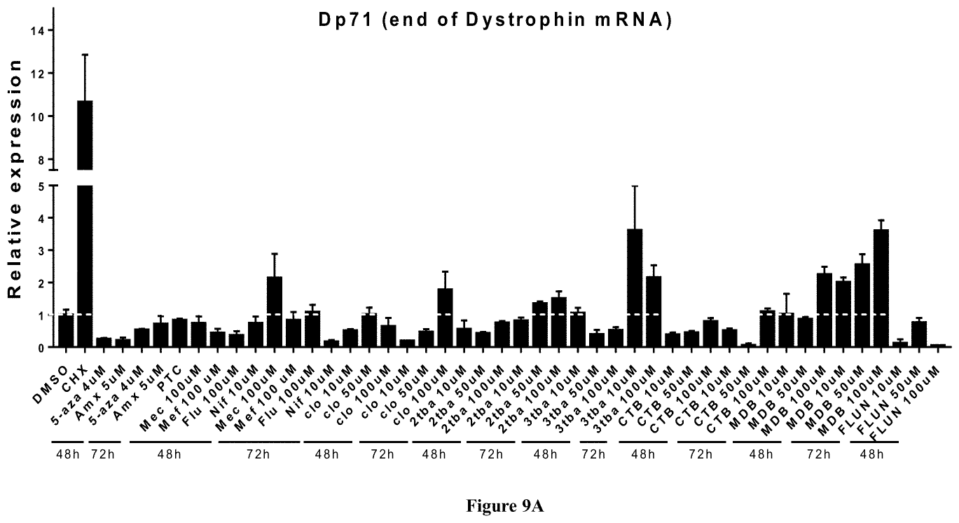

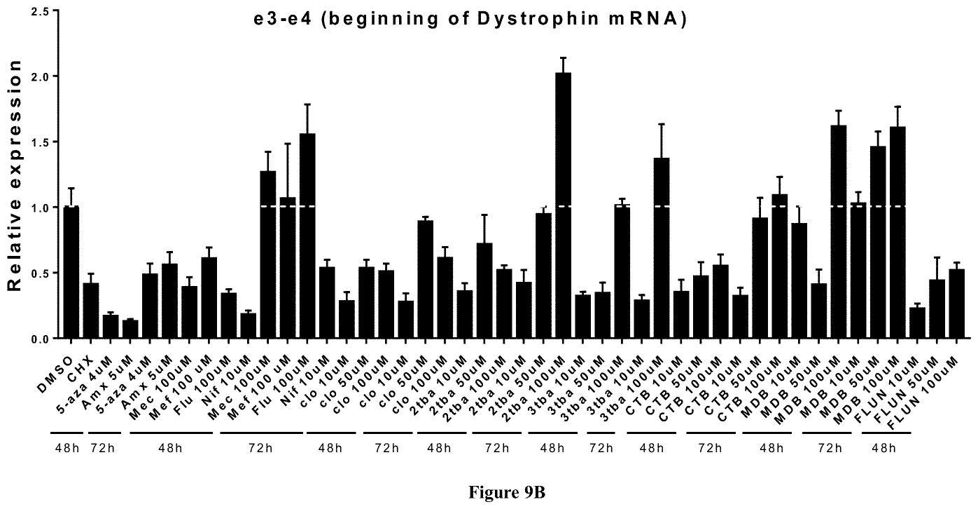

Though these meclofenamic acid derived compounds all showed positive results, the effect was not as high as might be desired. Thus, novel FTO/DMD targeting compounds were also tested. Primary skin fibroblasts derived from a DMD patient with a nonsense mutation in exon 53 of the dystrophin gene were exposed to compounds of FIGS. 7A-F. The positive control cycloheximide (9 ug/ml) produced a robust, greater than 10-fold, increase in dystrophin expression and Mec also produced a more modest 2-3-fold increase after 72 hours (FIG. 9A). Two compounds, 4-chloro-2-[3-(trifluoromethyl)anilino]benzoic acid (CTB) and Flunixin Meglumine (Flun) essentially had no effect on dystrophin levels. Two others, Clonixin (Clo) and 2-(2-toluidino)benzoic acid (2TBA), had a modest effect after 72 hours and at higher dose. Importantly, two other compounds, 2-(3-toluidino)benzoic acid (3TBA), methyl 10,11-dihydro-5H-dibenzo[b,f]azepine-4-carboxylate (MDB) had effects that were significantly superior to that of meclofenamic acid (FIG. 9A). 3TBA produced a 4-fold increase after 72 hours even at a dose of only 10 uM. After only 48 hours MDB produced a comparable effect to meclofenamic acid's effect after 72 hours and was effective after 72 hours at all doses. The 100 uM dose in particular produced a 4-fold increase comparable to 10 uM 3TBA. These results were acquired using primers than amplify dystrophin near its 3′ end (exons 65-66, DP71). Primers that detect dystrophin nears its 5′ end (exons 3-4) produced similar results (FIG. 9B).

At 72 hours, Mec, Mef and Flu all produced an increase in mRNA expression, though Flu did not. Flun similarly had not effect, as was observed in the cells from the first patient. CTB at high dose showed a possible increase although it was within the error. Unlike with the first patient, Clo did not produce an increase in mRNA expression, however, unexpectedly 2TBA was highly effective and a dose of 100 uM after 72 hours there was a 2-fold increase in mRNA expression, the highest observed for any compound (FIG. 9B). 3TBA was once again effective although the response was not as positive as for the first patient's cells, and MDB was once again a superior option, as it was effective at a dose of 100 uM even after 48 hours, and at 72 hours was effective at all doses (the 100 uM test was removed due to technical problems).

Stabilization of other NMD targets (SRSF6 and ATF3) was also examined in fibroblast cells from a subject with a nonsense mutation in exon 53 (FIG. 10A-B). Splicing factor SRSF6 was not increased by Mec or any of its derivatives, however, 2TBA and 3TBA and to a lesser extend MDB did produce increased expression (FIG. 10A). Interestingly, the effects of 2TBA and 3TBA were strongest after only 48 hours and with the lowest dose (10 uM). ATF3 mRNA expression was increased by Mec and its derivatives at 48 hours, but not at 72, expect for Mef (FIG. 10B). MDB, 2TBA and 3TBA produced increased expression at both time points, while Clo and CTB for the most part only produced an increase at 48 hours (100 um Clo was comparable to Mef at 72 hours). These results, taken together, indicate that MDB, 2TBA and 3TBA were the most effective FTO inhibitory compounds.

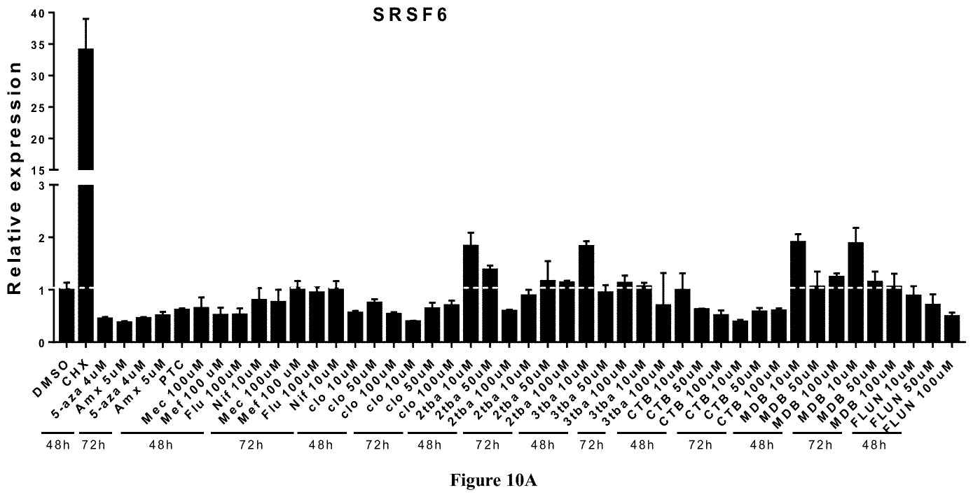

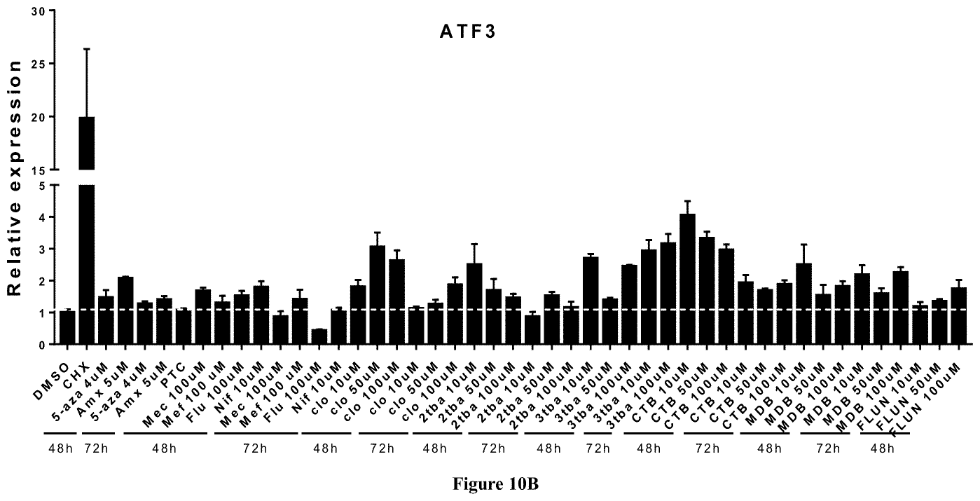

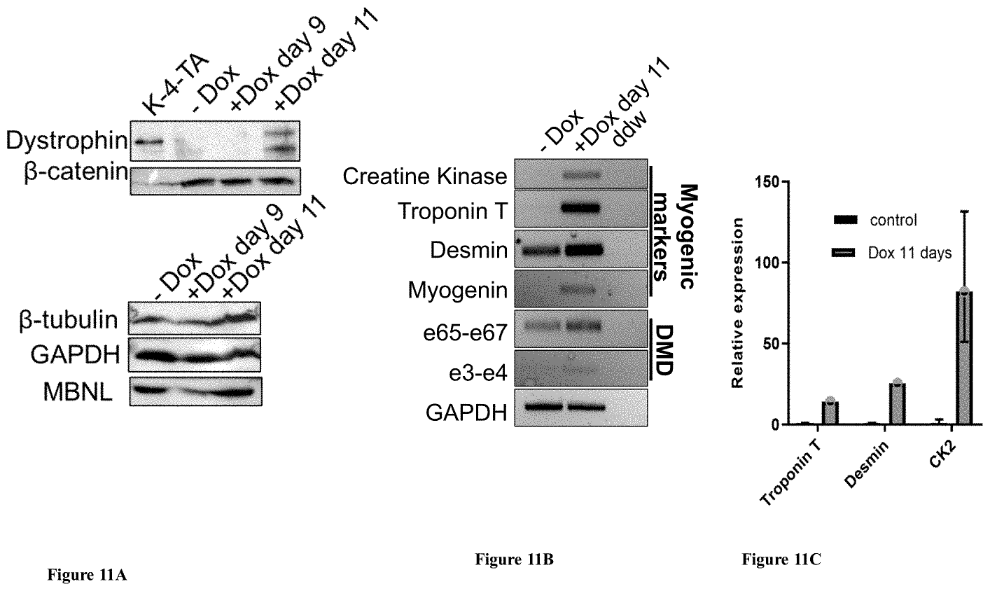

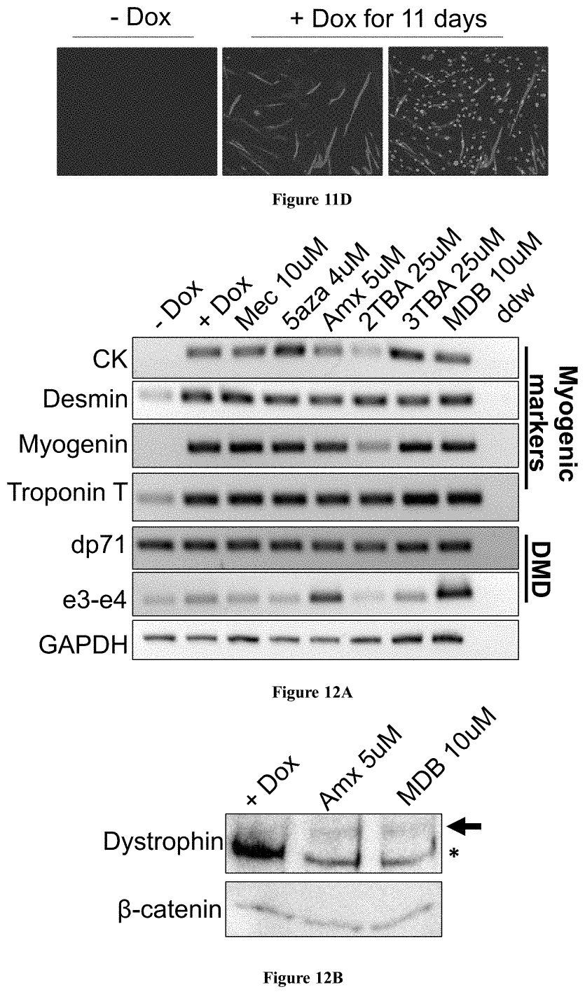

Since dystrophin protein is not detected in skin fibroblasts, a trans-differentiation system where patient-derived skin fibroblasts were transduced with a tet-inducible lentivirus expressing MyoD was enacted. Following 11 days of induction of MyoD by doxycycline treatment (FIG. 11D), the fibroblasts undergo morphological changes and express several muscle-specific markers (FIG. 11B-C). Moreover, in skin fibroblasts from a healthy male, dystrophin protein is detected 11 days post-induction (FIG. 11A). These results allowed for testing pharmacological treatments to modulate dystrophin mRNA and protein levels in patient patient-derived cells. Specifically, this system was used to test the effect of various NMD and FTO inhibitors on dystrophin mRNA stabilization in differentiated BMD patient-derived cells.

Primary skin fibroblasts of BMD patient-derived skin fibroblasts (del 45-49) were transdifferentiated into myocytes. Following a 24 hour infection with tet-on inducible myoD virus, media was changed and cells were allowed to grow for 24 hours. At 48 hours the cells were seeded on Matrigel coated dished (2.5×10\hat{ }5 cells/well) and then exposed to doxycycline (3 ug/ul) for 11 days. Dox was changed every 2 days. The cells were grown in the presence of the various compounds (added at day 6 of differentiation). MDB produced a robust increase in dystrophin mRNA, while 3TBA produced a more modest increase (FIG. 12A). A read-through drug Amlexanox (AMX) also elevated mRNA expression. Both of these compounds also produced increased protein as well (FIG. 12B).

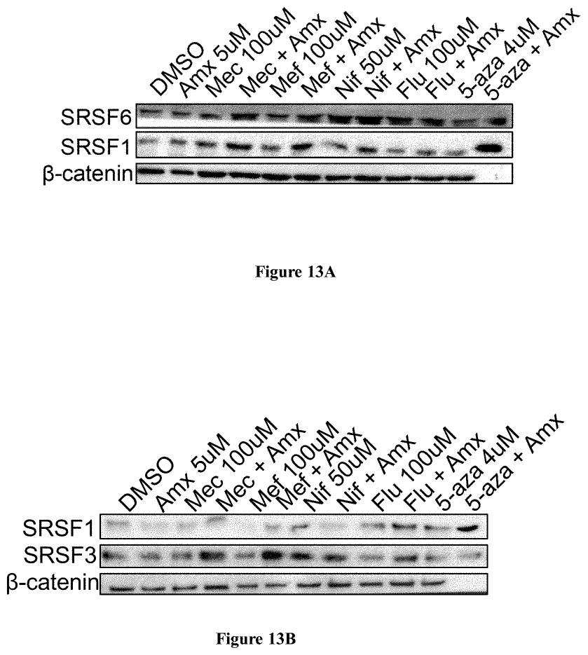

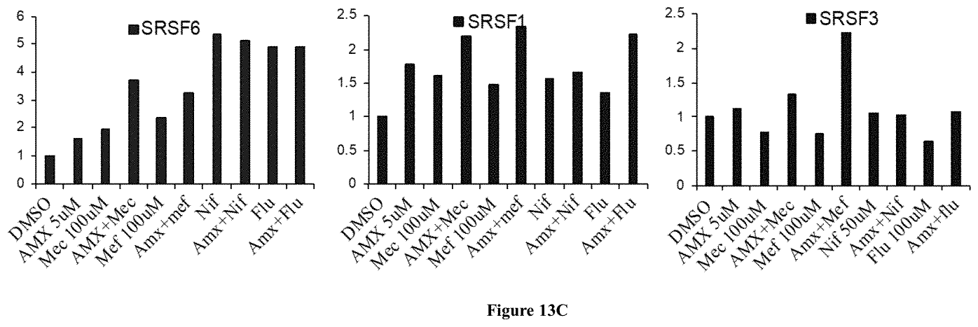

The use of read-through drugs for treating muscular dystrophies is well known in the art. Since both Amlexanox and MBD produced enhanced dystrophin expression, the effect of a combination of an FTO inhibitor with the read-through drug was tested. The expression of SR proteins in skin fibroblasts from BMD patients was evaluated by western blot. The protein expression was standardized to beta catenin and is summarized in FIGS. 13A-C. AMX, Mec, Mef, Nif and Flu all produced enhanced expression of SRSF6 and SRSF1 protein levels and the combinations of an FTO inhibitor and AMX often showing a combined higher effect (FIG. 13A-B). Unexpectedly, none of the compositions administered alone had a significant effect on SRSF3 protein expression, however, the combinations of Mec and AMX and Mef and AMX did enhance SRSF3 expression (FIG. 13C). These results underline the fact that modulation of enzymes involved in m⁶A biogenesis and specifically inhibition of FTO can enhance dystrophin protein levels and treat muscle related disease.

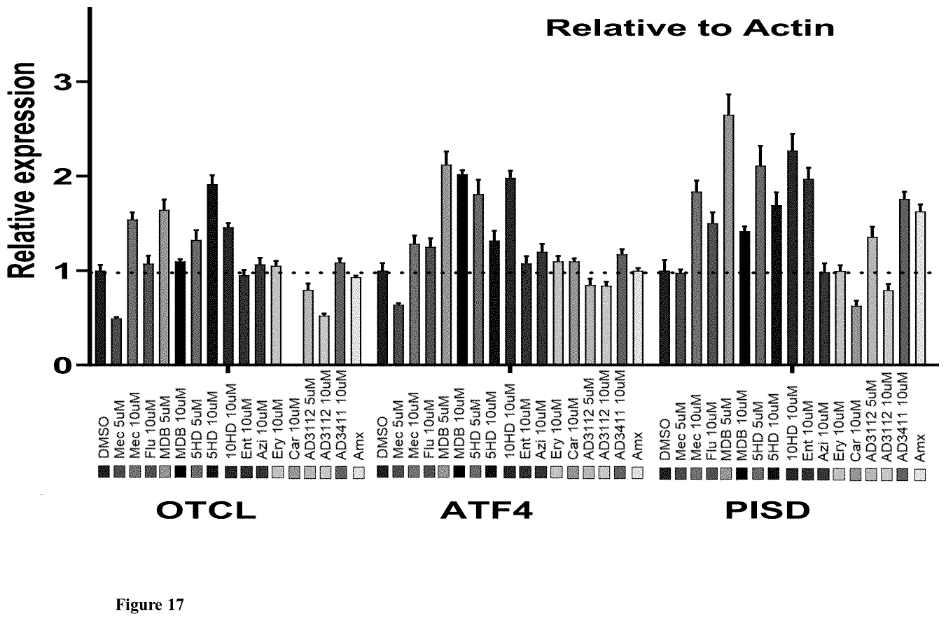

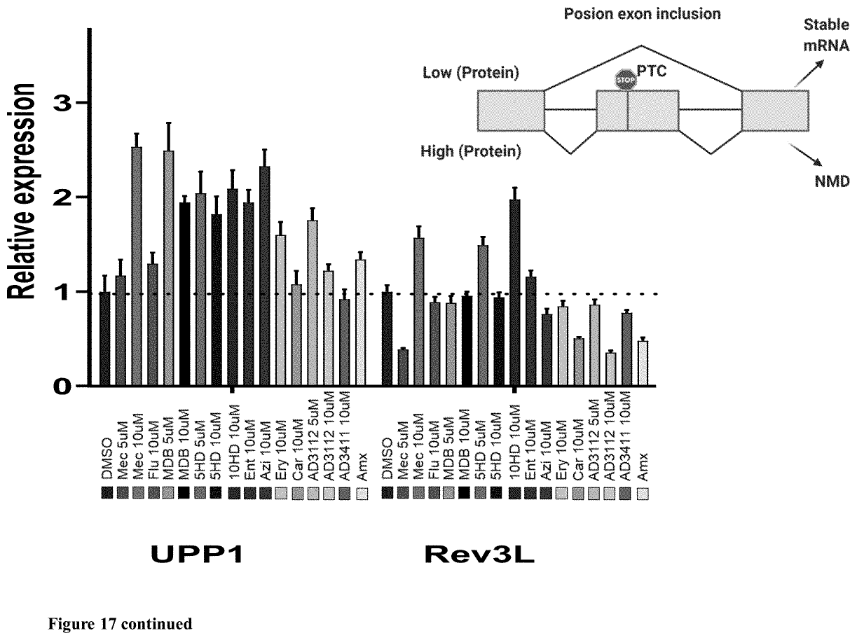

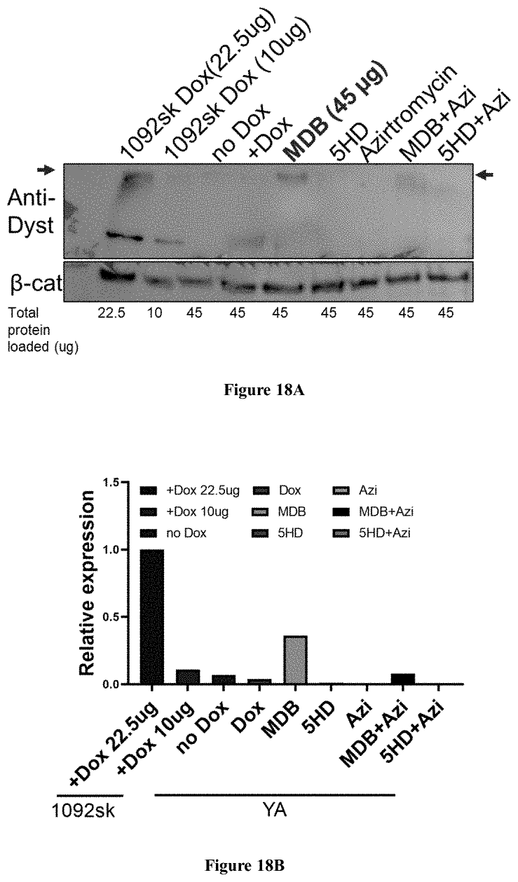

In order to identify additional FTO inhibitors that stabilize NMD-prone mRNAs, a drug screen was performed in HeLa cells. Expression of NMD-prone targets and Rev3L, a gene known to have a nonsense mutation in HeLa cells, was measured after treatment with various compounds. The screen identified already identified compound MDB and new compounds 5HD and 10HD (FIG. 17). Addition of the readthrough promoter azithromycin, as well as other readthrough promoting agents, did not enhance dystrophin expression. The use of BMD patient-derived cells allows for the detection of dystrophin protein levels, which is not possible in DMD patient-derived cells. Using BMD patient-derived fibroblasts (with duplication in exon 2-7) differentiated into muscle cells it was found that MDB markedly stabilizes dystrophin mRNA (FIG. 18D) and protein levels (FIG. 18A-B). In contrast, 5HD, and read through promoter azithromycin produced increases only on mRNA levels, although these increases were observed in many of the markers of muscle differentiation (FIG. 18C).

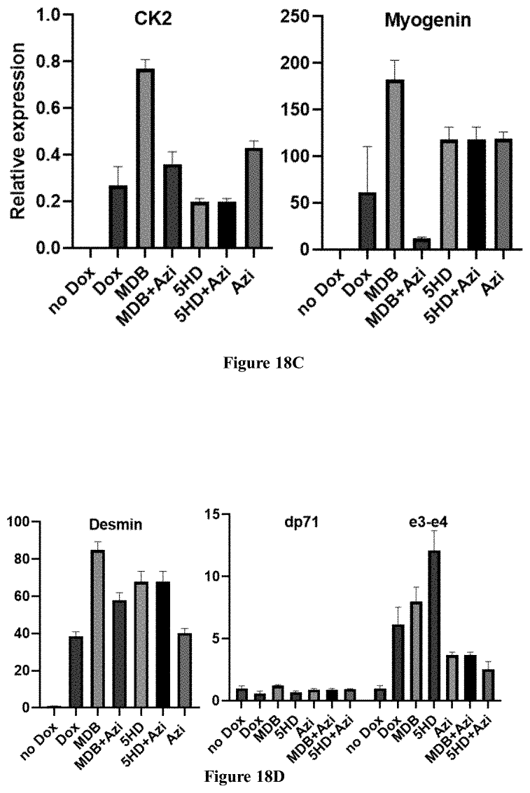

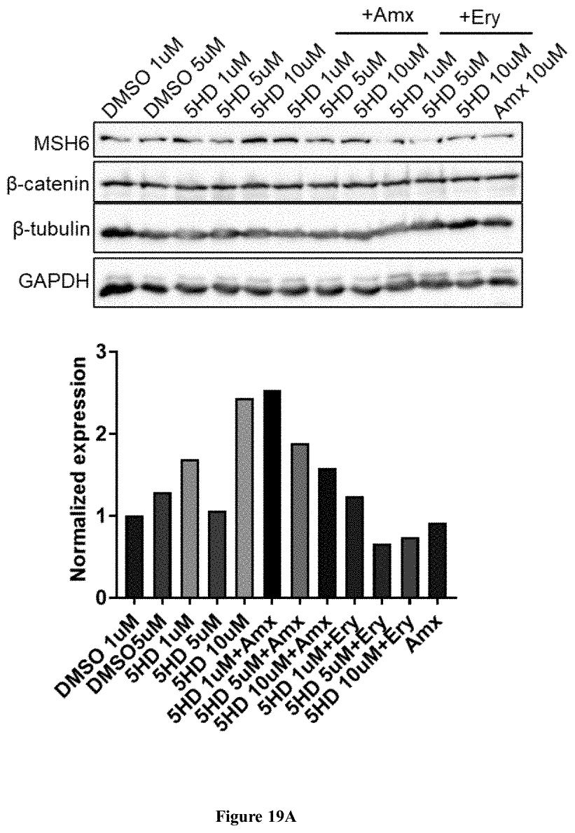

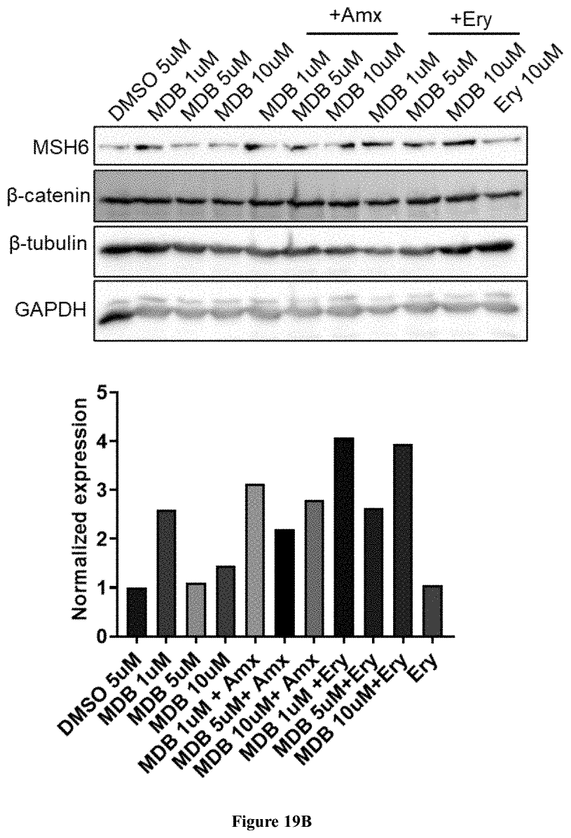

Ovca433 cells are an ovarian cancer cell line with a heterozygous nonsense mutation in the MSH6 gene. MSH6 is a gene in the mismatch repair pathway. Testing of both MDB and 5HD found that the compounds stabilized MSH6 protein levels in these cells (FIG. 19A-B) and that addition of read-through promoting AMX slightly enhanced the effect for both, while the promoter erythromycin was only effective when combined with MDB. This data suggest that FTO inhibitors can generally be used to stabilize identified mRNAs that are known to contain a nonsense mutation and that combination with a read-through promoter can enhance the effect.

### Example 3: FTO Inhibitors for Treating NMD-Associated Cancers

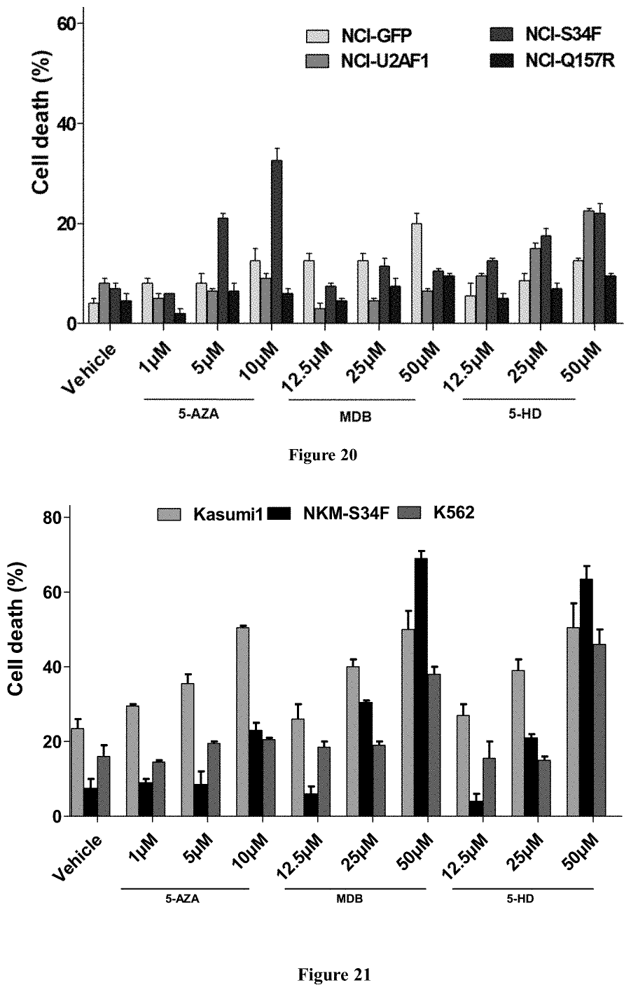

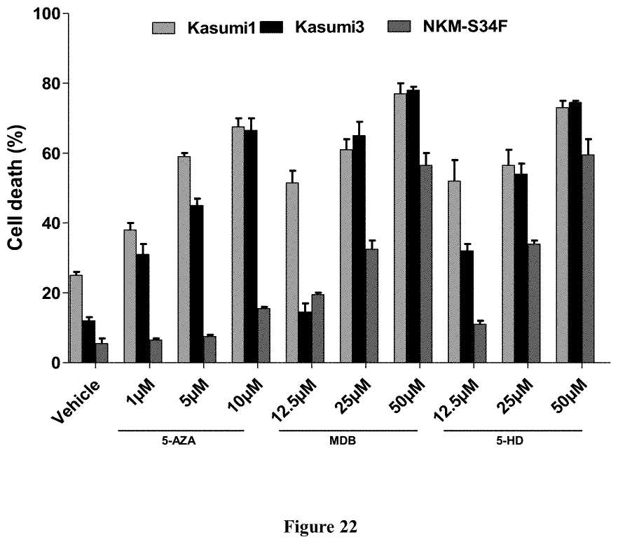

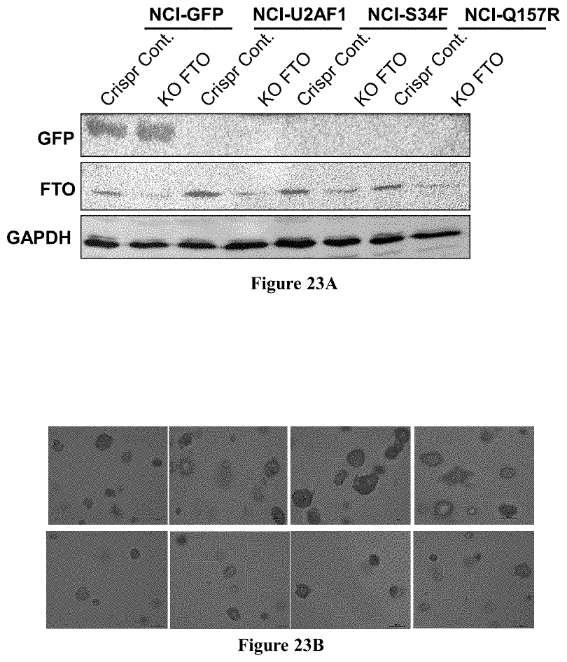

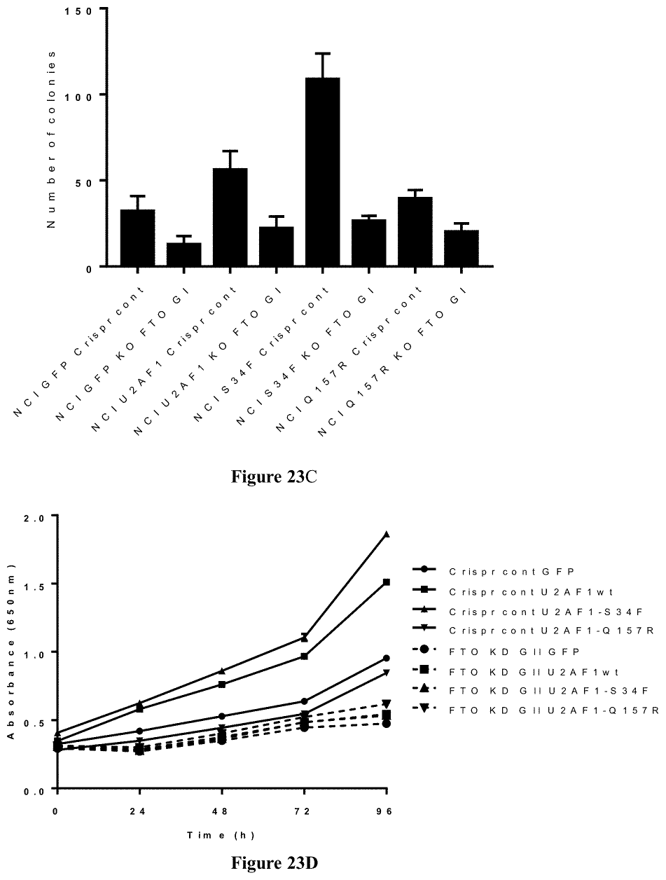

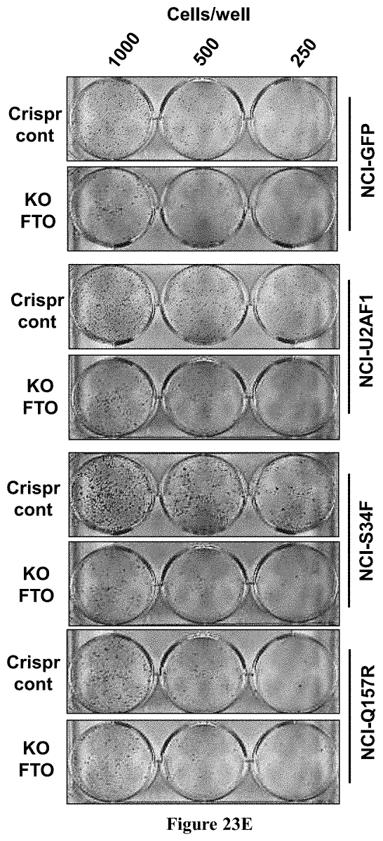

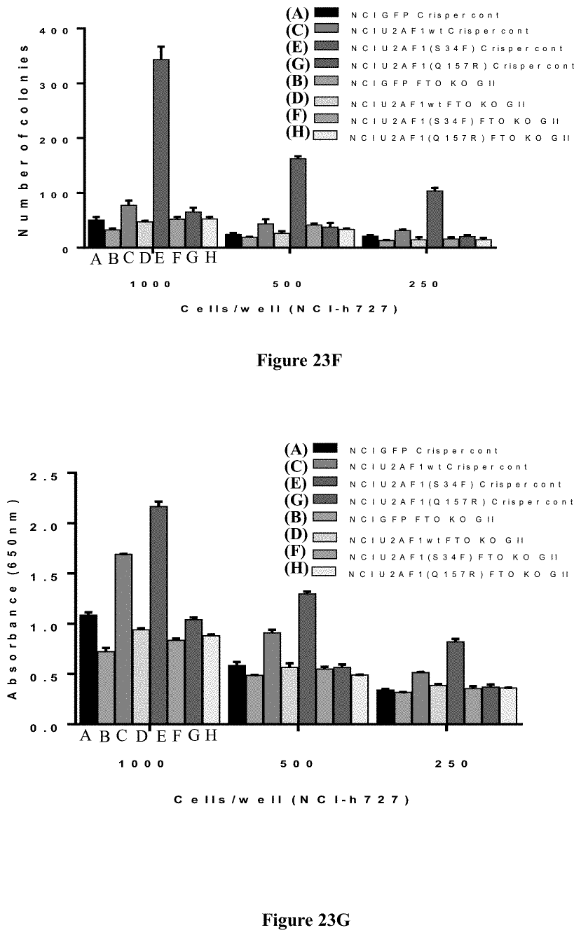

Mutations in the splicing factor U2AF1 are known to occur in both lung cancer and AML and promote oncogenesis. The effect of this mutation on the sensitivity of these cells to chemotherapy was tested. Four lung cancer cell lines were tested. NCI-GFP contained no mutation and was used as a negative control. Three lines contained a mutation. Of those lines, lung cancer cell line NCI-S34F, which contains a common lung U2AF1 mutation known to cause abhorrent splicing, showed increased sensitivity to 5-azacytidine, a known inhibitor of NMD (FIG. 20). 5-HD also produced an increase is cell death. Interestingly, when various AML cell lines were tested the presence of the S34F mutation in the U2AF1 gene produced increased resistance to 5-azacytidine (a common AML treatment) as compared to two lines without the mutation; the opposite effect of what was observed in the lung cells. A similar result was observed with a second chemotherapeutic: cisplatin. It was hypothesized that mutation in U2AF1, which causes the production of many aberrantly spliced transcripts, makes the cancer cells sensitive to NMD inhibition. Indeed, FTO inhibitors MDB and 5-HD both produced robust cell killing that was highest in the S34F cells (FIG. 21). Kasumi 1, an AML cell line, and K562, a CML cell line, both express wild type U2AF1. While the FTO inhibitors did increase cell death in these cell lines, the effect was not as pronounced as for the mutation bearing cells. These results demonstrate that cancer causing mutations that require effective NMD for cell survival, such as splicing factor and DNA repair mutations can also be targeted by FTO inhibitors.

1. A method of treating a disease characterized by mRNA instability or nonsense mediated decay (NMD) of an mRNA of a disease-associated gene in a subject in need thereof, the method comprising administering said subject a pharmaceutical composition comprising at least one agent that inhibits fat mass and obesity associated protein (FTO) expression or function, wherein said agent is not a non-steroidal anti-inflammatory drug (NSAID), thereby treating said disease.

2. (canceled)

3. (canceled)

4. The method of claim 1, wherein said agent is not meclofenamic acid or a derivative thereof selected from Mefenamic acid, Niflumic acid, and Flufenamic acid.

5. (canceled)

6. (canceled)

7. The method of claim 1, wherein said agent is not an isooxazoline derivative.

8. The method of claim 1, wherein said agent is a small molecule FTO inhibitor.

9. The method of claim 1, wherein said agent is a nucleic acid molecule that inhibits FTO transcription, inhibits FTO translation, induces FTO mRNA degradation or alters the FTO genetic locus.

10. (canceled)

11. The method of claim 1, wherein said non-NSAID agent is an FTO inhibitor selected from the group consisting of: 2-(2-toluidino)benzoic acid (2TBA); 2-(3-toluidino)benzoic acid (3TBA); 4-chloro-2-[3-(trifluoromethyl)anilino]benzoic acid (CTB); 5H-Dibenz[b,f]azepine (5HD), Clonixin, 10H-Dibenz[b,f]azepine (10HD), and methyl 10,11-dihydro-5H-dibenzo[b,f]azepine-4-carboxylate (MDB).

12. The method of claim 11, wherein said FTO inhibitor is selected from MDB, 2TBA, 3TBA and 5HD.

13. (canceled)

14. The method of claim 1, wherein mRNA instability comprises aberrant mRNA degradation.

15. The method of claim 1, wherein the disease is further characterized by the presence of a premature termination codon.

16. The method of claim 1, wherein said disease is selected from a muscular dystrophy characterized by mRNA instability or NMD of a muscle promoting gene and cancer characterized by mRNA instability or NMD of an anti-cancer gene.

17. The method of claim 1, wherein the disease is selected from the group consisting of: muscular dystrophy, cystic fibrosis, Ullrich disease, factor VII deficiency, Hailey-Hailey disease, hemophilia A, hemophilia B, leucocyte adhesion deficiency 1 (LAD1), cancer, McArdle disease, obesity and pathological conditions related to bone-mineral density disorders.

18. The method of claim 17, wherein said muscular dystrophy is selected from Bechet's muscular dystrophy, and Duchenne muscular dystrophy, and said cancer is selected from lung cancer and acute myeloid leukemia (AML).

19. The method of claim 17, wherein said cancer does not comprise oncogenic FTO expression, comprises a mutation of a splicing factor gene or a DNA repair gene, optionally wherein the DNA repair gene is a mismatch repair (MMR) gene, or both.

20. (canceled)

21. The method of claim 1, further comprising confirming mRNA instability or NMD of said mRNA of said disease-associated gene before said administering.

22. The method of claim 1, wherein said disease is characterized by NMD and further comprises administering at least one read-through promoting agent.

23. The method of claim 22, wherein the read-through promoting agent is selected from the group consisting of: aminoglycosides, modified aminoglycosides, erythromycin, azithromycin, (5Z)-2-Amino-5-[[5-(2-nitrophenyl)-2-furanyl]methylene]-4(5H)-thiazolone (RTC13), 3-[5-(2-Fluorophenyl)-1,2,4-oxadiazol-3-yl]benzoic acid (Ataluren), and 2-amino-7-isopropyl-5-oxo-5H-chromeno[2,3-b]pyridine-3-carboxylic acid (Amlexanox).

24. (canceled)

25. A kit comprising at least one agent that decreases FTO expression, function or both and at least one read-through promoting agent.

26. A method of determining suitability of a subject suffering from a disease to be treated by a method of claim 1, the method comprising measuring mRNA stability of an mRNA of a gene associated with said disease in said subject, wherein determining instability of said mRNA indicates said subject is suitable for treatment by said method.

27. The method of claim 26, wherein said measuring mRNA stability comprises:
a. measuring NMD in said subject and wherein detecting NMD indicates said subject is suitable for treatment
b. receiving a sample from said subject and measuring mRNA stability in said sample; or
c. both.

28. (canceled)

29. (canceled)

30. (canceled)

31. The kit of claim 25, comprising a pharmaceutical composition comprising said at least one agent that decreases FTO expression, function or both, said at least one read-through promoting agent and a pharmaceutically acceptable carrier


SEQUENCE LISTING (34 sequences)

SEQ ID NO: 1 - DNA, 21 bp, Homo sapiens
cagcatattgagaacctcttc

SEQ ID NO: 2 - DNA, 19 bp, Homo sapiens
ctgcaaaacccgcagtgcc

SEQ ID NO: 3 - DNA, 23 bp, Homo sapiens
ctggctgctgaatgtttatgata

SEQ ID NO: 4 - DNA, 20 bp, Homo sapiens
cttgccacttgcttgaaaag

SEQ ID NO: 5 - DNA, 20 bp, Homo sapiens
gccattggagagctgtcttc

SEQ ID NO: 6 - DNA, 20 bp, Homo sapiens
gggccatctggaacataaga

SEQ ID NO: 7 - DNA, 18 bp, Homo sapiens
ggcattgtgggctacgtg

SEQ ID NO: 8 - DNA, 18 bp, Homo sapiens
cttcaggagcagagcaga

SEQ ID NO: 9 - DNA, 20 bp, Homo sapiens
tgagcttgacaaagtggtcg

SEQ ID NO: 10 - DNA, 20 bp, Homo sapiens
ggctctccagaacatcatcc

SEQ ID NO: 11 - DNA, 21 bp, Homo sapiens
gcttgagtcatggaaggaggg

SEQ ID NO: 12 - DNA, 19 bp, Homo sapiens
ggaggtctttggccaactg

SEQ ID NO: 13 - DNA, 23 bp, Homo sapiens
cttctcagtcctccccaggacac

SEQ ID NO: 14 - DNA, 25 bp, Homo sapiens
cttgtaaactcttactgtctaatcc

SEQ ID NO: 15 - DNA, 20 bp, Homo sapiens
gggctacaaacccactgaca

SEQ ID NO: 16 - DNA, 20 bp, Homo sapiens
aacgtgtagcccttgatgct

SEQ ID NO: 17 - DNA, 20 bp, Homo sapiens
agcggaagaaggaggaagag

SEQ ID NO: 18 - DNA, 20 bp, Homo sapiens
gttcgcgttccttctcagtt

SEQ ID NO: 19 - DNA, 21 bp, Homo sapiens
agcgcagaattgaatctctca

SEQ ID NO: 20 - DNA, 20 bp, Homo sapiens
acctgctgttcctgaagctg

SEQ ID NO: 21 - DNA, 16 bp, Homo sapiens
ggtgcccagcgaatgc

SEQ ID NO: 22 - DNA, 19 bp, Homo sapiens
tgatgctgtccacgatgga

SEQ ID NO: 23 - DNA, 25 bp, Artificial
  Synthetic
caccgctgatcagaagccagaatgt

SEQ ID NO: 24 - DNA, 25 bp, Artificial
  Synthetic
aaacacattctggcttctgatcagc

SEQ ID NO: 25 - DNA, 25 bp, Artificial
  Synthetic
caccggagttgattgaggtaaagcg

SEQ ID NO: 26 - DNA, 25 bp, Artificial
  Synthetic
aaaccgctttacctcaatcaactcc

SEQ ID NO: 27 - DNA, 25 bp, Artificial
  Synthetic
caccgcatcagggtctgccttgcgg

SEQ ID NO: 28 - DNA, 25 bp, Artificial
  Synthetic
aaacccgcaaggcagaccctgatgc

SEQ ID NO: 29 - DNA, 46 bp, Artificial
  Synthetic
ggggaagaattcaccagccaccatggatgaagcgcaccccgactgc

SEQ ID NO: 30 - DNA, 59 bp, Artificial
  Synthetic
ttccccgtcgactcaagcgtaatctggaacatcgtatgggtagggttttgcttccagaa

SEQ ID NO: 31 - DNA, 46 bp, Artificial
  Synthetic
ggggaagaattcaccagccaccatggatggcggccgccagcggcta

SEQ ID NO: 32 - DNA, 62 bp, Artificial
  Synthetic
ttccccgtcgactcaagcgtaatctggaacatcgtatgggtaaaatatattagatttggttt

SEQ ID NO: 33 - DNA, 72 bp, Artificial
  Synthetic
ggggaatacgtaaccagccaccatgtacccatacgatgttccagattacgcttcggacacgtggagctctat

SEQ ID NO: 34 - DNA, 34 bp, Artificial
  Synthetic
ggggaagtcgacctataaattcttaggtttagag